SEQUENCE LISTING (6 sequences)

SEQ ID NO: 1 - protein, 136 aa, Artificial Sequence
  Synthetic
EVQLVESGGGLVQAGGSLRLSCAASGRTFSSYAMGWFRQAPGKEREFVVAINWSSGSTYYADSVKGRFTISRDNAKNTMYLQMNSLKPEDTAVYYCAAGYQINSGNYNFKDYEYDYWGQGTQVTVSSALEHHHHHH

SEQ ID NO: 2 - protein, 1317 aa, Artificial Sequence
  Synthetic
MTADKLVFFVNGRKVVEKNADPETTLLAYLRRKLGLSGTKLGCGEGGCGACTVMLSKYDRLQNKIVHFSANACLAPICSLHHVAVTTVEGIGSTKTRLHPVQERIAKSHGSQCGFCTPGIVMSMYTLLRNQPEPTMEEIENAFQGNLCRCTGYRPILQGFRTFARDGGCCGGDGNNPNCCMNQKKDHSVSLSPSLFKPEEFTPLDPTQEPIFPPELLRLKDTPRKQLRFEGERVTWIQASTLKELLDLKAQHPDAKLVVGNTEIGIEMKFKNMLFPMIVCPAWIPELNSVEHGPDGISFGAACPLSIVEKTLVDAVAKLPAQKTEVFRGVLEQLRWFAGKQVKSVASVGGNIITASPISDLNPVFMASGAKLTLVSRGTRRTVQMDHTFFPGYRKTLLSPEEILLSIEIPYSREGEYFSAFKQASRREDDIAKVTSGMRVLFKPGTTEVQELALCYGGMANRTISALKTTQRQLSKLWKEELLQDVCAGLAEELHLPPDAPGGMVDFRCTLTLSFFFKFYLTVLQKLGQENLEDKCGKLDPTFASATLLFQKDPPADVQLFQEVPKGQSEEDMVGRPLPHLAADMQASGEAVYCDDIPRYENELSLRLVTSTRAHAKIKSIDTSEAKKVPGFVCFISADDVPGSNITGICNDETVFAKDKVTCVGHIIGAVVADTPEHTQRAAQGVKITYEELPAIITIEDAIKNNSFYGPELKIEKGDLKKGFSEADNVVSGEIYIGGQEHFYLETHCTIAVPKGEAGEMELFVSTQNTMKTQSFVAKMLGVPANRIVVRVKRMGGGFGGKETRSTVVSTAVALAAYKTGRPVRCMLDRDEDMLITGGRHPFLARYKVGFMKTGTVVALEVDHFSNVGNTQDLSQSIMERALFHMDNCYKIPNIRGTGRLCKTNLPSNTAFRGFGGPQGMLIAECWMSEVAVTCGMPAEEVRRKNLYKEGDLTHFNQKLEGFTLPRCWEECLASSQYHARKSEVDKFNKENCWKKRGLCIIPTKFGISFTVPFLNQAGALLHVYTDGSVLLTHGGTEMGQGLHTKMVQVASRALKIPTSKIYISETSTNTVPNTSPTAASVSADLNGQAVYAACQTILKRLEPYKKKNPSGSWEDWVTAAYMDTVSLSATGFYRTPNLGYSFETNSGNPFHYFSYGVACSEVEIDCLTGDHKNLRTDIVMDVGSSLNPAIDIGQVEGAFVQGLGLFTLEELHYSPEGSLHTRGPSTYKIPAFGSIPIEFRVSLLRDCPNKKAIYASKAVGEPPLFLAASIFFAIKDAIRAARAQHTGNNVKELFRLDSPATPEKIRNACVDKFTTL

SEQ ID NO: 3 - protein, 10 aa, Artificial Sequence
  Synthetic
GGGGSGGGGS

SEQ ID NO: 4 - protein, 1463 aa, Artificial Sequence
  Synthetic
MTADKLVFFVNGRKVVEKNADPETTLLAYLRRKLGLSGTKLGCGEGGCGACTVMLSKYDRLQNKIVHFSANACLAPICSLHHVAVTTVEGIGSTKTRLHPVQERIAKSHGSQCGFCTPGIVMSMYTLLRNQPEPTMEEIENAFQGNLCRCTGYRPILQGFRTFARDGGCCGGDGNNPNCCMNQKKDHSVSLSPSLFKPEEFTPLDPTQEPIFPPELLRLKDTPRKQLRFEGERVTWIQASTLKELLDLKAQHPDAKLVVGNTEIGIEMKFKNMLFPMIVCPAWIPELNSVEHGPDGISFGAACPLSIVEKTLVDAVAKLPAQKTEVFRGVLEQLRWFAGKQVKSVASVGGNIITASPISDLNPVFMASGAKLTLVSRGTRRTVQMDHTFFPGYRKTLLSPEEILLSIEIPYSREGEYFSAFKQASRREDDIAKVTSGMRVLFKPGTTEVQELALCYGGMANRTISALKTTQRQLSKLWKEELLQDVCAGLAEELHLPPDAPGGMVDFRCTLTLSFFFKFYLTVLQKLGQENLEDKCGKLDPTFASATLLFQKDPPADVQLFQEVPKGQSEEDMVGRPLPHLAADMQASGEAVYCDDIPRYENELSLRLVTSTRAHAKIKSIDTSEAKKVPGFVCFISADDVPGSNITGICNDETVFAKDKVTCVGHIIGAVVADTPEHTQRAAQGVKITYEELPAIITIEDAIKNNSFYGPELKIEKGDLKKGFSEADNVVSGEIYIGGQEHFYLETHCTIAVPKGEAGEMELFVSTQNTMKTQSFVAKMLGVPANRIVVRVKRMGGGFGGKETRSTVVSTAVALAAYKTGRPVRCMLDRDEDMLITGGRHPFLARYKVGFMKTGTVVALEVDHFSNVGNTQDLSQSIMERALFHMDNCYKIPNIRGTGRLCKTNLPSNTAFRGFGGPQGMLIAECWMSEVAVTCGMPAEEVRRKNLYKEGDLTHFNQKLEGFTLPRCWEECLASSQYHARKSEVDKFNKENCWKKRGLCIIPTKFGISFTVPFLNQAGALLHVYTDGSVLLTHGGTEMGQGLHTKMVQVASRALKIPTSKIYISETSTNTVPNTSPTAASVSADLNGQAVYAACQTILKRLEPYKKKNPSGSWEDWVTAAYMDTVSLSATGFYRTPNLGYSFETNSGNPFHYFSYGVACSEVEIDCLTGDHKNLRTDIVMDVGSSLNPAIDIGQVEGAFVQGLGLFTLEELHYSPEGSLHTRGPSTYKIPAFGSIPIEFRVSLLRDCPNKKAIYASKAVGEPPLFLAASIFFAIKDAIRAARAQHTGNNVKELFRLDSPATPEKIRNACVDKFTTLGGGGSGGGGSEVQLVESGGGLVQAGGSLRLSCAASGRTFSSYAMGWFRQAPGKEREFVVAINWSSGSTYYADSVKGRFTISRDNAKNTMYLQMNSLKPEDTAVYYCAAGYQINSGNYNFKDYEYDYWGQGTQVTVSSALEHHHHHH

SEQ ID NO: 5 - protein, 1463 aa, Artificial Sequence
  Synthetic
EVQLVESGGGLVQAGGSLRLSCAASGRTFSSYAMGWFRQAPGKEREFVVAINWSSGSTYYADSVKGRFTISRDNAKNTMYLQMNSLKPEDTAVYYCAAGYQINSGNYNFKDYEYDYWGQGTQVTVSSALEHHHHHHGGGGSGGGGSMTADKLVFFVNGRKVVEKNADPETTLLAYLRRKLGLSGTKLGCGEGGCGACTVMLSKYDRLQNKIVHFSANACLAPICSLHHVAVTTVEGIGSTKTRLHPVQERIAKSHGSQCGFCTPGIVMSMYTLLRNQPEPTMEEIENAFQGNLCRCTGYRPILQGFRTFARDGGCCGGDGNNPNCCMNQKKDHSVSLSPSLFKPEEFTPLDPTQEPIFPPELLRLKDTPRKQLRFEGERVTWIQASTLKELLDLKAQHPDAKLVVGNTEIGIEMKFKNMLFPMIVCPAWIPELNSVEHGPDGISFGAACPLSIVEKTLVDAVAKLPAQKTEVFRGVLEQLRWFAGKQVKSVASVGGNIITASPISDLNPVFMASGAKLTLVSRGTRRTVQMDHTFFPGYRKTLLSPEEILLSIEIPYSREGEYFSAFKQASRREDDIAKVTSGMRVLFKPGTTEVQELALCYGGMANRTISALKTTQRQLSKLWKEELLQDVCAGLAEELHLPPDAPGGMVDFRCTLTLSFFFKFYLTVLQKLGQENLEDKCGKLDPTFASATLLFQKDPPADVQLFQEVPKGQSEEDMVGRPLPHLAADMQASGEAVYCDDIPRYENELSLRLVTSTRAHAKIKSIDTSEAKKVPGFVCFISADDVPGSNITGICNDETVFAKDKVTCVGHIIGAVVADTPEHTQRAAQGVKITYEELPAIITIEDAIKNNSFYGPELKIEKGDLKKGFSEADNVVSGEIYIGGQEHFYLETHCTIAVPKGEAGEMELFVSTQNTMKTQSFVAKMLGVPANRIVVRVKRMGGGFGGKETRSTVVSTAVALAAYKTGRPVRCMLDRDEDMLITGGRHPFLARYKVGFMKTGTVVALEVDHFSNVGNTQDLSQSIMERALFHMDNCYKIPNIRGTGRLCKTNLPSNTAFRGFGGPQGMLIAECWMSEVAVTCGMPAEEVRRKNLYKEGDLTHFNQKLEGFTLPRCWEECLASSQYHARKSEVDKFNKENCWKKRGLCIIPTKFGISFTVPFLNQAGALLHVYTDGSVLLTHGGTEMGQGLHTKMVQVASRALKIPTSKIYISETSTNTVPNTSPTAASVSADLNGQAVYAACQTILKRLEPYKKKNPSGSWEDWVTAAYMDTVSLSATGFYRTPNLGYSFETNSGNPFHYFSYGVACSEVEIDCLTGDHKNLRTDIVMDVGSSLNPAIDIGQVEGAFVQGLGLFTLEELHYSPEGSLHTRGPSTYKIPAFGSIPIEFRVSLLRDCPNKKAIYASKAVGEPPLFLAASIFFAIKDAIRAARAQHTGNNVKELFRLDSPATPEKIRNACVDKFTTL

SEQ ID NO: 6 - protein, 5 aa, Artificial Sequence
  Synthetic
  REPEAT (1)..(5): may be repeated 1 or more times
GGGGS

(19) United States
(12) Patent Application Publication
(10) Pub. No.: US 2025/0205355 A1
(43) Pub. Date: Jun. 26, 2025

(54) METHODS AND SYSTEMS FOR APPLICATIONS INVOLVING ANTIGEN-PRESENTING CELLS

(71) Applicant: Viska Bio, Inc., Cambridge, MA (US)

(72) Inventor: Eric T. Fossel, Cambridge, MA (US)

(73) Assignee: Viska Bio, Inc., Cambridge, MA (US)

(21) Appl. No.: 18/848,612

(22) PCT Filed: Mar. 23, 2022
(86) PCT No.: PCT/US2022/021520

§ 371 Date: Sep. 19, 2024

(51) Int. Cl. A61K 47/68; A61K 38/44; A61K 45/06; C07K 16/10; C07K 16/18

(52) U.S. Cl. CPC A61K 47/6815; A61K 38/443; A61K 45/06; A61K 47/6841; A61K 47/6843; C07K 16/1002; C07K 16/1045; C07K 16/18; C12Y 101/03004; C12Y 111/01; C12Y 117/03002; C07K 2317/569; C07K 2317/92

(57) ABSTRACT

The present disclosure generally relates to systems and methods for treating and/or reversing a disease or condition such as aging, nonalcoholic steatohepatitis (NASH), neurodegenerative disease such as Alzheimer's and Parkinson's, obesity pulmonary fibrosis, congestive heart failure, or cancer using immunogenic cell death, e.g., lysosome-induced immunogenic cell death. In certain embodiments, the application is directed towards compositions that comprise an antibody and an enzyme capable of producing reactive oxygen species. In some cases, the lysosome of cells associated with the disease or condition may be targeted by the composition for induced cell death.

### TECHNICAL FIELD

The present disclosure generally relates to systems and methods for treating a disease or condition such as aging, Nonalcoholic Steatohepatitis (NASH) disease, neurodegenerative diseases such as Alzheimer's and Parkinson's disease, obesity, pulmonary fibrosis, congestive heart failure, and cancer by targeted apoptosis of particular cells (e.g., senescent cells, fat cells, fibroblasts, or cancer cells) associated with the disease or condition.

### BACKGROUND

Diseases and conditions such as aging, Nonalcoholic Steatohepatitis (NASH) disease, neurodegenerative diseases such as Alzheimer's and Parkinson's disease, obesity, pulmonary fibrosis, congestive heart failure, and cancer may be multifactorial in their pathogenesis. Treatments of these diseases or conditions are needed.

### SUMMARY

The present disclosure generally relates to systems and methods for treating and/or reversing a disease or condition such as aging, nonalcoholic steatohepatitis (NASH), neurodegenerative disease such as Alzheimer's and Parkinson's, obesity pulmonary fibrosis, congestive heart failure, or cancer using immunogenic cell death, e.g., lysosome-induced immunogenic cell death. In certain embodiments, the application is directed towards compositions that comprise an antibody and an enzyme capable of producing reactive oxygen species. In some cases, the lysosome of cells associated with the disease or condition may be targeted by the composition for induced cell death.

The subject matter of the present disclosure involves, in some cases, interrelated products, alternative solutions to a particular problem, and/or a plurality of different uses of one or more systems and/or compositions.

For example, in certain embodiments, by targeting cells that are likely contributors to a disease or condition, such as senescent cells, fat cells, fibroblasts, or cancer cells, the disease or condition may be treated through removal of the cells. Such removal can be accomplished by targeted immunogenic cell death, in accordance with some embodiments.

In another aspect, the disclosure is directed towards a composition for treating a disease or condition. In some embodiments, the composition comprises: a fusion protein comprising an enzyme and at least a portion of an antibody, wherein the at least a portion of the antibody is configured to bind to a cell associated with a disease or condition, and wherein the enzyme is configured to increase lysosomal membrane permeability of the cell.

In an aspect, the disclosure is directed towards a method of treating a disease or condition. In some embodiments, the method comprises: administering to a subject a composition comprising a fusion protein that comprises an enzyme and at least a portion of an antibody, wherein the at least a portion of the antibody recognizes a cell associated with the disease or condition of the subject, and wherein the enzyme is configured to increase lysosomal membrane permeability of the cell.

In one aspect, the disclosure is directed towards a composition. In some embodiments, the composition comprises: an antibody that recognizes a senescent cell, a fat cell, a myocardial fibroblast, or a pulmonary fibroblast, and an enzyme that is configured to increase lysosomal membrane permeability of the recognized senescent cell, fat cell, myocardial fibroblast, or pulmonary fibroblast.

In another aspect, the disclosure is directed towards a composition comprising: an antibody that recognizes a senescent cell, a fat cell, a myocardial fibroblast, or a pulmonary fibroblast, and an enzyme that is able to create reactive oxygen species.

In still another aspect, the disclosure is directed towards a method, comprising: administering, to a subject, a composition comprising an antibody that recognizes a senescent cell, a fat cell, a myocardial fibroblast, or a pulmonary fibroblast, and an enzyme that is able to increase lysosomal membrane permeability of the recognized senescent cell, fat cell, myocardial fibroblast, or pulmonary fibroblast.

In yet another aspect, the disclosure is directed towards a method, comprising: administering, to a subject, a composition comprising an antibody that recognizes a senescent cell, a fat cell, a myocardial fibroblast, or a pulmonary fibroblast, and an enzyme that is able to create reactive oxygen species.

Several methods are disclosed herein of administering a subject with a composition for treatment of a particular disease or condition. It is to be understood that in each such aspect of the disclosure specifically includes, also, the composition for use in the treatment of that particular disease or condition, as well as use of the composition for the manufacture of a medicament for the treatment of that particular disease or condition.

Other advantages and novel features of the present disclosure will become apparent from the following detailed description of various non-limiting embodiments of the disclosure when considered in conjunction with the accompanying figures. In cases where the present specification and a document incorporated by reference include conflicting and/or inconsistent disclosure, the present specification shall control.

### BRIEF DESCRIPTION OF THE DRAWINGS

Non-limiting embodiments of the present disclosure will be described by way of example with reference to the accompanying figures, which are schematic and are not intended to be drawn to scale unless otherwise indicated. In the figures, each identical or nearly identical component illustrated is typically represented by a single numeral. For purposes of clarity, not every component is labeled in every figure, nor is every component of each embodiment of the disclosure shown where illustration is not necessary to allow those of ordinary skill in the art to understand the disclosure. In the figures:

FIG. 1 presents an exemplary mechanism for lysosome induced immunogenic cell death induced by reactive oxygen species, according to some embodiments; and

FIG. 2 presents a perspective, schematic illustration of an exemplary fusion protein comprising an exemplary nanobody that is coupled to an exemplary protein associated with a disease or condition, according to some embodiments.

### DETAILED DESCRIPTION

The present disclosure generally relates to systems and methods for treating or reversing a disease or condition (such as aging, Nonalcoholic Steatohepatitis (NASH) disease, neurodegenerative diseases such as Alzheimer's and Parkinson's disease, obesity, pulmonary fibrosis, congestive heart failure, or cancer) through immunogenic cell death, e.g., lysosome-induced immunogenic cell death.

This disclosure includes multiple aspects, which may be used separately or together. For example, in one aspect, the disclosure is directed towards preparation of the body and/or brain of a subject for treatment, for example, by withdrawal and suppression of antioxidants, supply of n-3 through n-6 and other unsaturated fatty acids, and/or treatment of the subject with statins.

In one aspect, the disclosure is directed towards a composition comprising an enzyme. For example, the composition may comprise an enzyme and an antibody. In some embodiments, the enzyme is connected to the antibody. In some cases the antibody and enzyme may be expressed as a single fusion protein, as discussed in greater detail below.

The enzyme may be a naturally occurring or synthetically modified enzyme or a fragment thereof. In some embodiments, the enzyme is an oxidoreductase. For example, the enzyme may be oxidase or a peroxidase in various embodiments. Other exemplary oxidoreductases that may be used include, but are not limited to those described below.

Oxidases, according to some embodiments, are enzymes that catalyze an oxidation-reduction reaction, including reactions involving oxygen (O₂) as an electron acceptor. The oxygen may be reduced to water (H₂O) or hydrogen peroxide (H₂O₂).

Non-limiting examples of oxidases include glucose oxidase, monoamine oxidase, cytochrome P450 oxidase, NADPH oxidase, xanthine oxidase, L-gulonolactone oxidase, laccase, lysyl oxidase, or the like. Enzymes described herein may be recombinant enzymes.

Peroxidases, according to some embodiments, are enzymes that act on substrates such as hydrogen peroxide or lipid peroxides. Non-limiting examples include horseradish peroxidase, cytochrome c peroxidase, ascorbate peroxidase, chloride peroxidase, glutathione peroxidase, haloperoxidase, lactorperoxidase, manganese peroxidase, myeloperoxidase, thyroid peroxidase, vanadium boromoperoxidase, or the like.

In some embodiments, the enzyme is an oxidase or a peroxidase suitable for the creation of antigens as described herein.

In some embodiments, the composition can be administered to a subject. The subject may also be provided with a substrate to the enzyme. The composition may be used separately or together with other compositions. In some embodiments, the composition is used to treat and/or cure a disease or condition such as aging, Nonalcoholic Steatohepatitis (NASH) disease, neurodegenerative disease such as Alzheimer's and Parkinson's disease, obesity, pulmonary fibrosis, congestive heart failure, or cancer. For example, the enzyme may be connected to an antibody that recognizes a cell associated with a disease or condition (e.g., a senescent cell, a fat cell, a fibroblast), as described in greater detail below. In some cases, the composition may target a lysosome of the cell(s). Targeting the lysosome of the cell may induce cell death of the cell associated with the disease or condition (e.g., the senescent cell, the fat cell, the fibroblast), in some embodiments.

The enzyme may be an oxidase, a peroxidase, or other enzyme that is able to create reactive oxygen species. The composition may be administered to a subject, for example, via infusion or other administration techniques such as those described herein. The subject may also be provided with a substrate to the enzyme in some cases. Without wishing to be bound by any theory, it is believed that the antibody helps to localize the composition to cells associated with a chosen disease or condition. Once localized, the substrate of the enzyme may be administered to the subject, e.g., infused, which may set off a series of events that result in attack of the lysosome membrane, release of lysosomal contents into the cell and cell death through apoptosis. Thus, in some embodiments the disclosure is directed towards lysosome-induced immunogenic cell death, LIICD.

In some cases, a substrate for the enzyme may also be administered to the subject. The substrate and the enzyme may be administered in any suitable order, e.g., sequentially and/or simultaneously. Non-limiting examples of substrates include hypoxanthine or xanthine for xanthine oxidase, or glucose for glucose oxidase. As additional examples, monoamine oxidase may act on serotonin, melatonin, norepinephrine, epinephrine, phenethylamine, benzylamine, dopamine, tyramine, tryptamine, etc. for monoamine oxidase; NADPH for NADPH peroxidase; ascorbate for ascorbate peroxidase; or the like.

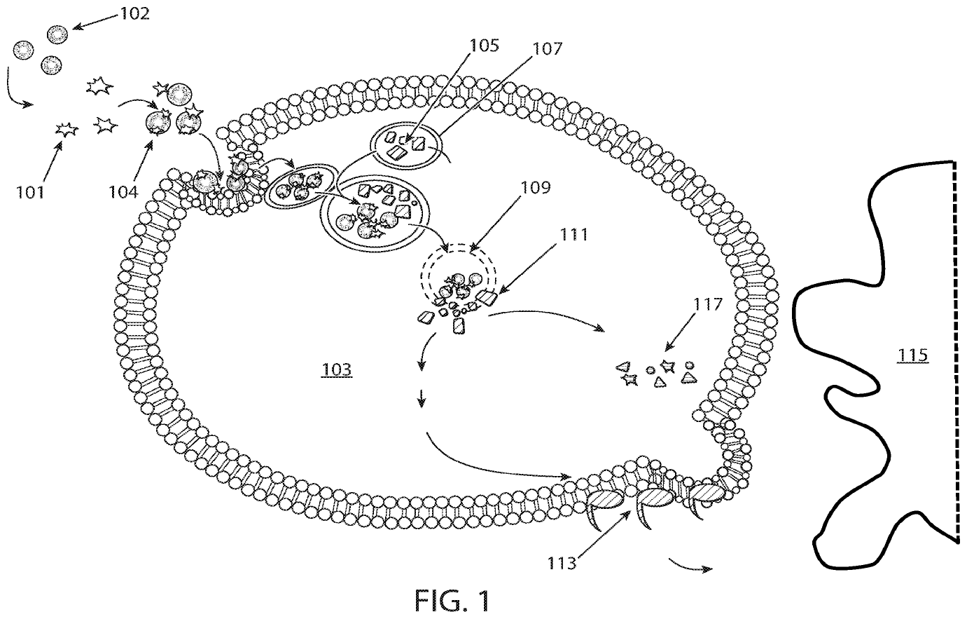

Without wishing to be bound by any theory, it is believed that certain enzymes (e.g., oxidases and peroxidases) create reactive oxygen species such as superoxides, peroxides, hydroxyl radicals, etc., by oxidizing a substrate associated with the enzyme. Accordingly, in some embodiments, a substrate of an enzyme may be optionally added (e.g., may be included with the composition, or may be administered to a subject to be treated with the composition) to facilitate such a reaction. In some embodiments, the reactive oxygen species are endocytosed by a targeted cell. The reactive oxygen species may interact with a membrane of a targeted cell. In some embodiments, oxidation of unsaturated lipids can cause their endocytosis. FIG. 1 presents a cross-sectional schematic diagram of this process, in which reactive oxygen species 101 react with unsaturated lipids 102, creating oxidized unsaturated lipids 104 that are endocytosed into cell 103. Without wishing to be bound by theory, the interaction between the lysosome of the targeted cell and the oxidized unsaturated lipids may induce a lysosomal membrane of the cell to become more permeable. For example, in FIG. 3, oxidized unsaturated lipids 104 are endocytosed into cell 103, where lipids 104 are taken up by lysosome 105, causing lysosome membrane 107 to become permeable lysosome membrane 109. Thus, an enzyme may be configured to increase lysosomal membrane permeability of a cell, e.g., by creating reactive oxygen species. Changes in lysosomal membrane permeability may be detected one of ordinary skill using a galectin puncta assay.

Increased lysosomal membrane permeability may cause the cell to leak degradative enzymes. For example, referring again to FIG. 1, once lysosome membrane 107 becomes permeable lysosome membrane 109, degradative enzymes 111 can leak through permeable lysosome membrane 109. Destabilization of the cell's lysosomal membrane may disperse lysosomal enzymes throughout the cell. The cell may then create cell-specific neoantigens that can be recognized by the immune system, and/or that may trigger apoptosis and/or cell death. This is presented in FIG. 1, where the leakage of degradative enzymes 111 creates degradation byproducts 117 and independently causes cell 103 to express neoantigens 113, signaling cell 103 for endocytosis by dendritic cell 115. Thus, reactive oxygen species may be useful in some embodiments to cause lysosome-induced immunogenic cell death (LIICD). Accordingly, it is believed that cells associated with diseases or conditions such as aging, Nonalcoholic Steatohepatitis (NASH) disease, neurodegenerative disease such as Alzheimer's and Parkinson's disease, obesity, pulmonary fibrosis, congestive heart failure, or cancer may be targeted (e.g., with an antibody) for the delivery of enzymes that can kill the cells via administration of a composition as described herein.

In addition, it should be understood that in some cases, a drug may be used within the composition, instead and/or in addition to an enzyme that creates a reactive oxygen species.

In some embodiments, the disclosure is directed towards a composition comprising an enzyme and an antibody or antibody fragment. The antibody or antibody fragment may be selected to recognize a cell (e.g., a senescent cell, a fat cell, a cancer cell, or a fibroblast) associated with a disease or condition of a subject. The antibody or antibody fragment may be connected to the enzyme, e.g., via a linkage, such as a covalent bond. In some embodiments, the composition comprises a fusion protein comprising the enzyme and the antibody or antibody fragment, as described in greater detail below. For example, in some embodiments, a composition as described herein may create antigens, e.g., by increase lysosomal membrane permeability and/or leakage, and/or by creating reactive oxygen species, etc., as discussed herein. The creation of antigens may allow the immune system of the subject, e.g., T cells, to recognize the cell (e.g., the senescent cell, the fat cell, a cancer cell, or the fibroblast) associated with the disease or condition of the subject by recognizing the antigens created within the cell. In some embodiments, recognition by T cells may result in lysosome-induced immunogenic cell death of the cell. In some embodiments, the immunogenic cell death of the cell may train an immune response against target cells (e.g., senescent cells, fat cells, fibroblasts, or cancer cells), resulting in long-term treatment of the disease or condition as a result of an ongoing immune response.

In some embodiments, the composition may be used to treat the condition of aging. For example, the enzyme of the composition is configured to induce death of senescent cells, in some embodiments. Without wishing to be bound by theory, senescent cells may be incapable of cell division, and/or may be associated with diseases of aging.

Herein, it has been inventively recognized that through the use of compositions comprising enzymes and senescent cell recognizing antibodies, senescent cells may be targeted for immunogenic cell death in some embodiments. The immunogenic cell death of senescent cells may reduce the quantity or proportion of senescent cells within the body. The reduction in the quantity or proportion of senescent cells may, in turn, retard or reverse the aging process.

According to certain embodiments, a composition useful for treating aging comprises at least a portion of an antibody (e.g., an entire antibody or an antibody fragment) that recognizes senescent cells. For example, the composition may comprise at least a portion of canakinumab, an anti-DPP4 antibody, an anti-CD9 antibody, or another of a variety of appropriate antibodies that recognize senescent cells and are known to those of ordinary skill in the art.

The at least a portion of the antibody (e.g., the entire antibody or the antibody fragment) and the enzyme may be connected to one another, as discussed in greater detail below. In some embodiments, the enzyme and the antibody form a fusion protein. The fusion protein may be particularly advantageous for treating aging. In some embodiments, for example, the fusion protein may bind to senescent cells with a higher affinity than would be expected from an identical enzyme connected to an identical antibody or antibody portion by a linker. In some embodiments, the fusion protein also has a longer lifetime in the subject, which may also result in an improved performance of the composition.

Senescent cells have also been implicated in diseases such as nonalcoholic steatohepatitis (NASH). In some embodiments, the composition may be used to treat the NASH. For example, as described above with reference to aging, the enzyme of the composition may be configured to induce death of senescent cells.

Herein, it has been inventively recognized that through the use of compositions comprising enzymes and senescent cell recognizing antibodies, senescent cells may be targeted for immunogenic cell death in some embodiments. The immunogenic cell death of senescent cells may reduce the quantity or proportion of senescent cells within the body. The reduction in the quantity or proportion of senescent cells may, in turn, retard or reverse NASH.

As discussed above with reference to aging, according to certain embodiments, a composition useful for treating NASH comprises at least a portion of an antibody (e.g., an entire antibody or an antibody fragment) that recognizes senescent cells.

The at least a portion of the antibody (e.g., the entire antibody or the antibody fragment) and the enzyme may be connected to one another, as discussed in greater detail below. In some embodiments, the enzyme and the antibody form a fusion protein. The fusion protein may be particularly advantageous for treating NASH. In some embodiments, for example, the fusion protein may bind to senescent cells with a higher affinity than would be expected from an identical enzyme connected to an identical antibody or antibody portion by a linker. In some embodiments, the fusion protein also has a longer lifetime in the subject, which may also result in an improved performance of the composition.

Senescent cells also contribute to neurodegenerative diseases such as Alzheimer's disease and Parkinson's disease. In some embodiments, the composition may be used to treat neurodegenerative diseases. For example, as described above with reference to aging, the enzyme of the composition may be configured to induce death of senescent cells.

Herein, it has been inventively recognized that through the use of compositions comprising enzymes and senescent cell recognizing antibodies, senescent cells may be targeted for immunogenic cell death in some embodiments. The immunogenic cell death of senescent cells may reduce the quantity or proportion of senescent cells within the body. The reduction in the quantity or proportion of senescent cells may, in turn, retard or reverse neurodegenerative disease.

As discussed above with reference to aging, according to certain embodiments, a composition useful for treating neurodegenerative disease comprises at least a portion of an antibody (e.g., an entire antibody or an antibody fragment) that recognizes senescent cells.

The at least a portion of the antibody (e.g., the entire antibody or the antibody fragment) and the enzyme may be connected to one another, as discussed in greater detail below. In some embodiments, the enzyme and the antibody form a fusion protein. The fusion protein may be particularly advantageous for treating neurodegenerative diseases. In some embodiments, for example, the fusion protein may bind to senescent cells with a higher affinity than would be expected from an identical enzyme connected to an identical antibody or antibody portion by a linker. In some embodiments, the fusion protein also has a longer lifetime in the subject, which may also result in an improved performance of the composition.

In some embodiments, the composition may be used to treat the disease of obesity. For example, the enzyme of the composition may be configured to be connected to a fat cell (also known as an adipocyte). An overabundance of fat cells and/or an abundance of over-sized fat cells may be associated with obesity, as well as a number of related conditions, such as insulin resistance/diabetes.

Herein, it has been inventively recognized that through the use of compositions comprising enzymes and fat cell recognizing antibodies, fat cells may be targeted for immunogenic cell death in some embodiments. The immunogenic cell death of fat cells may reduce the quantity or proportion of fat cells within the body. The reduction in the quantity or proportion of fat cells may, in turn, retard or reverse obesity.

According to certain embodiments, a composition useful for treating obesity comprises at least a portion of an antibody (e.g., an entire antibody or an antibody fragment) that recognizes fat cells. For example, the composition may comprise at least a portion of an adipocyte-specific IgG antibody, an anti-ASC-1 antibody, an anti-PAT2 antibody, an anti-P2RX5 antibody, an adipocyte-type fatty acid-binding protein (A-FABP), or another of a variety of appropriate antibodies that recognize fat cells and are known to those of ordinary skill in the art.

The at least a portion of the antibody (e.g., the entire antibody or the antibody fragment) and the enzyme may be connected to one another, as discussed in greater detail below. In some embodiments, the enzyme and the antibody form a fusion protein. The fusion protein may be particularly advantageous for treating obesity. In some embodiments, for example, the fusion protein may bind to fat cells with a higher affinity than would be expected from an identical enzyme connected to an identical antibody or antibody portion by a linker. In some embodiments, the fusion protein also has a longer lifetime in the subject, which may also result in an improved performance of the composition.

In some embodiments, the composition may be used to treat the disease of pulmonary fibrosis. For example, the enzyme of the composition may be configured to be connected to a fibroblast (e.g., a pulmonary fibroblast). An overabundance of fibroblasts (e.g., resulting from fibroblast proliferation) in lung tissue may be associated with pulmonary fibrosis.

Herein, it has been inventively recognized that through the use of compositions comprising enzymes and fibroblast recognizing antibodies (e.g., pulmonary fibroblast recognizing antibodies), fibroblasts (e.g., pulmonary fibroblasts) may be targeted for immunogenic cell death in some embodiments. The immunogenic cell death of fibroblasts may reduce the quantity or proportion of fibroblasts within the lungs of a subject. The reduction in the quantity or proportion of fibroblasts may, in turn, retard or reverse pulmonary fibrosis.

According to certain embodiments, a composition useful for treating pulmonary fibrosis comprises at least a portion of an antibody (e.g., an entire antibody or an antibody fragment) that recognizes fibroblasts. For example, the composition may comprise at least a portion of fibroblast antibody TE-7, fibroblast antibody ER-TR7, or monoclonal antibody fibroblast 1B10, an anti-CD82 antibody, an anti-a-SMA antibody, an anti-ITG-P1 antibody, or another of a variety of appropriate antibodies that recognize fibroblasts and are known to those of ordinary skill in the art.

The at least a portion of the antibody (e.g., the entire antibody or the antibody fragment) and the enzyme may be connected to one another, as discussed in greater detail below. In some embodiments, the enzyme and the antibody form a fusion protein. The fusion protein may be particularly advantageous for treating pulmonary fibrosis. In some embodiments, for example, the fusion protein may bind to fibroblasts (e.g., pulmonary fibroblasts) with a higher affinity than would be expected from an identical enzyme connected to an identical antibody or antibody portion by a linker. In some embodiments, the fusion protein also has a longer lifetime in the subject, which may also result in an improved performance of the composition.

In some embodiments, the composition may be used to treat the disease of congestive heart failure. For example, the enzyme of the composition may be configured to be connected to a fibroblast (e.g., a myocardial fibroblast). An overabundance of fibroblasts (e.g., resulting from fibroblast proliferation) in myocardial tissue may be associated with congestive heart failure.

Herein, it has been inventively recognized that through the use of compositions comprising enzymes and fibroblast recognizing antibodies (e.g., myocardial fibroblast recognizing antibodies), fibroblasts (e.g., myocardial fibroblasts) may be targeted for immunogenic cell death in some embodiments. The immunogenic cell death of fibroblasts may reduce the quantity or proportion of fibroblasts within the lungs of a subject. The reduction in the quantity or proportion of fibroblasts may, in turn, retard or reverse congestive heart failure.

According to certain embodiments, a composition useful for treating congestive heart failure comprises at least a portion of an antibody (e.g., an entire antibody or an antibody fragment) that recognizes fibroblasts, as described above.

The at least a portion of the antibody (e.g., the entire antibody or the antibody fragment) and the enzyme may be connected to one another, as discussed in greater detail below. In some embodiments, the enzyme and the antibody form a fusion protein. The fusion protein may be particularly advantageous for treating congestive heart failure. In some embodiments, for example, the fusion protein may bind to fibroblasts (e.g., myocardial fibroblasts) with a higher affinity than would be expected from an identical enzyme connected to an identical antibody or antibody portion by a linker. In some embodiments, the fusion protein also has a longer lifetime in the subject, which may also result in an improved performance of the composition.

In some embodiments, the composition may be used to treat the disease of cancer. For example, the enzyme of the composition may be configured to be connected to a cancer cell. Herein, it has been inventively recognized that through the use of compositions comprising enzymes and cancer cell recognizing antibodies, cancer cells may be targeted for immunogenic cell death in some embodiments. The immunogenic cell death of cancer cells may treat the cancer.

According to certain embodiments, a composition useful for treating cancer comprises at least a portion of an antibody (e.g., an entire antibody or an antibody fragment) that recognizes cancer cells. For example, the antibody may be an antibody for epidermal growth factor receptors (EGFR), which may be overexpressed in cancer cells, or may be an antibody to any of a variety of other antigens or other markers that are uniquely expressed or overexpressed in cancer cells. As a specific, example, the antibody may be (SEQ ID NO 1).

The at least a portion of the antibody (e.g., the entire antibody or the antibody fragment) and the enzyme may be connected to one another, as discussed in greater detail below. In some embodiments, the enzyme and the antibody form a fusion protein. The fusion protein may be particularly advantageous for treating cancer. In some embodiments, for example, the fusion protein may bind to cancer cells with a higher affinity than would be expected from an identical enzyme connected to an identical antibody or antibody portion by a linker. In some embodiments, the fusion protein also has a longer lifetime in the subject, which may also result in an improved performance of the composition.

The enzyme may be connected to another portion of the composition (e.g., an antibody or an antibody portion, etc.) covalently. In some embodiments, the enzyme and the other portion of the composition (e.g., an antibody or an antibody portion) are directly connected, such that the enzyme and the other portion form a single polypeptide chain. For example, the composition may be expressed as a single fusion protein containing both an enzyme (e.g. an oxidase, such as xanthine oxidase) and another portion of the composition (e.g., an antibody or an antibody portion). In other embodiments, however, other methods may be used to connect the enzyme to the composition, for example, directly bound to each other, or bound via one or more cross-linking agents. Non-limiting examples include glutaraldehyde, NHS-esters (N-hydroxysuccinimide) (e.g., dithiobis(succinimidylpropionate), dithiobis(sulfosuccinimidylpropionate), etc.), PEG groups, imidoesters (e.g., dimethyl adipimidate, dimethyl suberimidate, dimethyl pimelimidate, etc.), maleimides, pyridyls, carbodiimide, isocyanate, or the like. The antibody and the enzyme may be coupled through any suitable system, e.g., amine-to-amine, sulfhydryl-to-sulfhydryl, amine-to-sulfhydryl, carboxyl-to-amine, sulfhydryl-to-carbohydrate, hydroxyl-to-sulfhydryl, or the like. Those of ordinary skill in the art will be familiar with methods of cross-linking or conjugating proteins to each other.

A “fusion protein” generally refers to a hybrid polypeptide which comprises protein domains from at least two different proteins. One protein may be located at the amino-terminal (N-terminal) portion of the fusion protein or at the carboxy-terminal (C-terminal) protein thus forming an “amino-terminal fusion protein” or a “carboxy-terminal fusion protein,” respectively. A fusion protein may comprise different domains, for example, an enzyme and an antibody; or an enzyme and an antibody fragment. In some embodiments, the enzyme is fused at the N-terminus of the antibody or antibody fragment. In some embodiments, the enzyme is fused at the C-terminus of the antibody or antibody fragment.

In some embodiments, the enzyme is separated from the antibody or antibody fragment by a linker region. The linker region may be configured to separate the antibody from the enzyme spatially. In some embodiments, the linker region may have an amino acid sequence that comprises repeated subsequences. For example, in some embodiments the linker region has an amino acid sequence that comprises (GGGGS)ₙ (SEQ ID NO 6), where is an integer greater than or equal to 1 (e.g., 1, 2, 3, 4, 5, 6, 8, 10, or more). In some embodiments, the linker region is GGGGSGGGGS (SEQ ID NO 3). In other embodiments, the composition does not comprise a linker region.

In some embodiments, the fusion protein of the present disclosure comprises an amino acid sequence that is at least 70% identical to any one of SEQ ID NO: 4-5. For example, the fusion protein may comprise an amino acid sequence that is at least 70%, at least 80%, at least 90%, at least 95%, at least 99%, or at least 99.5% identical to any one of SEQ ID NOs: 4-5. In some embodiments, the fusion protein comprises an amino acid sequence that is 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.5% identical to any one of SEQ ID NOs: 4-5. In some embodiments, the fusion protein comprises the amino acid sequence of any one of SEQ ID NOs: 4-5. In some embodiments, the fusion protein consists of the amino acid sequence of any one of SEQ ID NOs: 4-5.

Further provided herein are enzyme variants and fusion proteins comprising such enzyme variants. In some embodiments, the fusion protein described herein comprises a modification. When the fusion protein is referred to herein, it encompasses all its variants and derivatives. Polypeptides comprising modifications have additional features other than amino acid contents. As used herein, a “modification” or “derivative” of a protein or polypeptide (e.g., the fusion protein described herein) produces a modified or derivatized polypeptide, which is a form of a given peptide that is chemically modified relative to the reference peptide, the modification including, but not limited to, oligomerization or polymerization, modifications of amino acid residues or peptide backbone, cross-linking, cyclization, conjugation, PEGylation, glycosylation, acetylation, phosphorylation, acylation, carboxylation, lipidation, thioglycolic acid amidation, alkylation, methylation, polyglycylation, glycosylation, polysialylation, adenylylation, PEGylation, fusion to additional heterologous amino acid sequences, or other modifications that substantially alter the stability, solubility, or other properties of the peptide while substantially retaining the activity of the polypeptides described herein. It is to be understood that the fusion protein comprising such modifications, are cross-linked, cyclized, conjugated, acylated, carboxylated, lipidated, acetylated, thioglycolic acid amidated, alkylated, methylated, polyglycylated, glycosylated, polysialylated, phosphorylated, adenylylated, PEGylated, or combination thereof. In some embodiments, the modified fusion protein of the present disclosure may contain non-amino acid elements, such as polyethylene glycols, lipids, poly- or mono-saccharide, and phosphates. The fusion protein of the present disclosure, may comprise the modifications disclosed herein at the C-terminus (e.g., C-terminal amidation), N-terminus (e.g., N-terminal acetylation). Terminal modifications are useful, and are well known, to reduce susceptibility to proteinase digestion, and therefore serve to prolong half-life of the polypeptides in solutions, particularly biological fluids where proteases may be present. In some embodiments, the fusion proteins described herein are further modified within the sequence, such as, modification by terminal-NH₂ acylation, e.g., acetylation, or thioglycolic acid amidation, by terminal-carboxylamidation, e.g., with ammonia, methylamine, and the like terminal modifications.

Terminal modifications are useful, to reduce susceptibility by proteinase digestion, and therefore can serve to prolong half-life of the polypeptides in solution, particularly in biological fluids where proteases may be present. Amino terminus modifications include methylation (e.g., —NHCH₃ or —N(CH₃)₂), acetylation (e.g., with acetic acid or a halogenated derivative thereof such as a-chloroacetic acid, a-bromoacetic acid, or a-iodoacetic acid), adding a benzyloxycarbonyl (Cbz) group, or blocking the amino terminus with any blocking group containing a carboxylate functionality defined by RCOO— or sulfonyl functionality defined by R—SO₂—, where R is selected from the group consisting of alkyl, aryl, heteroaryl, alkyl aryl, and the like, and similar groups. One can also incorporate a desamino acid at the N-terminus (so that there is no N-terminal amino group) to decrease susceptibility to proteases or to restrict the conformation of the polypeptide. In certain embodiments, the N-terminus is acetylated with acetic acid or acetic anhydride.

Carboxy terminus modifications include replacing the free acid with a carboxamide group or forming a cyclic lactam at the carboxy terminus to introduce structural constraints. One can also cyclize the peptides described herein, or incorporate a desamino or descarboxy residue at the termini of the peptide, so that there is no terminal amino or carboxyl group, to decrease susceptibility to proteases or to restrict the conformation of the peptide. Methods of circular peptide synthesis are known in the art. C-terminal functional groups of the peptides described herein include amide, amide lower alkyl, amide di(lower alkyl), lower alkoxy, hydroxy, and carboxy, and the lower ester derivatives thereof, and the pharmaceutically acceptable salts thereof.

In some embodiments, the fusion proteins described herein are phosphorylated. One can also readily modify peptides by phosphorylation, and other methods. In some embodiments, one can also replace the naturally occurring side chains of the genetically encoded amino acids (or the stereoisomeric D amino acids) with other side chains, for instance with groups such as alkyl, lower (C₁₋₆) alkyl, cyclic 4-, 5-, 6-, to 7-membered alkyl, amide, amide lower alkyl amide di(lower alkyl), lower alkoxy, hydroxy, carboxy and the lower ester derivatives thereof, and with 4-, 5-, 6-, to 7-membered heterocycles. For example, proline analogues in which the ring size of the proline residue is changed from 5 members to 4, 6, or 7 members can be employed.

Cyclic groups can be saturated or unsaturated, and if unsaturated, can be aromatic or non-aromatic. Heterocyclic groups preferably contain one or more nitrogen, oxygen, and/or sulfur heteroatoms. Examples of such groups include the furazanyl, furyl, imidazolidinyl, imidazolyl, imidazolinyl, isothiazolyl, isoxazolyl, morpholinyl (e.g. morpholino), oxazolyl, piperazinyl (e.g., 1-piperazinyl), piperidyl (e.g., 1-piperidyl, piperidino), pyranyl, pyrazinyl, pyrazolidinyl, pyrazolinyl, pyrazolyl, pyridazinyl, pyridyl, pyrimidinyl, pyrrolidinyl (e.g., 1-pyrrolidinyl), pyrrolinyl, pyrrolyl, thiadiazolyl, thiazolyl, thienyl, thiomorpholinyl (e.g., thiomorpholino), and triazolyl groups. These heterocyclic groups can be substituted or unsubstituted. Where a group is substituted, the substituent can be alkyl, alkoxy, halogen, oxygen, or substituted or unsubstituted phenyl.

In some embodiments, the fusion proteins described herein may be attached to one or more polymer moieties. In some embodiments, these polymers are covalently attached to the fusion proteins of the disclosure. In some embodiments, for therapeutic use of the end product preparation, the polymer is pharmaceutically acceptable. One skilled in the art will be able to select the desired polymer based on such considerations as whether the polymer-peptide conjugate will be used therapeutically, and if so, the desired dosage, circulation time, resistance to proteolysis, and other considerations.

All combinations of the different modifications and derivatizations are envisioned for the fusion protein described herein.

Other aspects of the present disclosure provide methods of producing the fusion protein. The fusion protein may be produced by expression form recombinant nucleic acids in appropriate cells (e.g., bacterial cell or eukaryotic cells) and isolated. To produce the fusion protein, nucleic acids encoding the fusion protein may be introduced to a cell (e.g., a bacterial cell or a eukaryotic cell such as a yeast cell or an insect cell. The cells may be cultured under conditions that allow the fusion protein to express from the nucleic acids encoding the fusion protein. Fusion proteins comprising a signal peptide can be secreted, e.g., into the culturing media and can subsequently be recovered. The fusion protein may be isolated using any methods of purifying a protein known in the art.

The nucleic acids encoding the fusion protein described herein may be obtained, and the nucleotide sequence of the nucleic acids determined, by any method known in the art. One skilled in the art is able to identify the nucleotide sequence encoding the fusion protein from the amino acid sequence of the fusion protein. The nucleic acids encoding the fusion protein of the present disclosure, may be DNA or RNA, double-stranded or single stranded. In some embodiments, the nucleotide sequence encoding the fusion protein may be codon optimized to adapt to different expression systems (e.g., for mammalian expression).

In some embodiments, the nucleic acid is comprised within a vector, such as an expression vector. In some embodiments, the vector comprises a promoter operably linked to the nucleic acid.

A variety of promoters can be used for expression of the fusion proteins described herein, including, but not limited to, cytomegalovirus (CMV) intermediate early promoter, a viral LTR such as the Rous sarcoma virus LTR, HIV-LTR, HTLV-1 LTR, the simian virus 40 (SV40) early promoter, E. coli lac UV5 promoter, and the herpes simplex tk virus promoter.

Regulatable promoters can also be used. Such regulatable promoters include those using the lac repressor from E. coli as a transcription modulator to regulate transcription from lac operator-bearing mammalian cell promoters, those using the tetracycline repressor (tetR), etc. Other systems include FK506 dimer, VP16 or p65 using astradiol, RU486, diphenol murislerone, or rapamycin.

Regulatable promoters that include a repressor with the operon can be used. In one embodiment, the lac repressor from Escherichia coli can function as a transcriptional modulator to regulate transcription from lac operator-bearing mammalian cell promoters. In one embodiment, a tetracycline inducible switch is used.

Additionally, the vector can contain, for example, some or all of the following: a selectable marker gene, such as the neomycin gene for selection of stable or transient transfectants in mammalian cells; enhancer/promoter sequences from the immediate early gene of human CMV for high levels of transcription; transcription termination and RNA processing signals from SV40 for mRNA stability; SV40 polyoma origins of replication and ColE1 for proper episomal replication; internal ribosome binding sites (IRESes), versatile multiple cloning sites; and T7 and SP6 RNA promoters for in vitro transcription of sense and antisense RNA. Suitable vectors and methods for producing vectors containing transgenes are well known and available in the art.

An expression vector comprising the nucleic acid can be transferred to a host cell by conventional techniques (e.g., electroporation, liposomal transfection, and calcium phosphate precipitation) and the transfected cells are then cultured by conventional techniques to produce the fusion proteins described herein. In some embodiments, the expression of the fusion proteins described herein is regulated by a constitutive, an inducible or a tissue-specific promoter.

The host cells used to express the fusion proteins described herein may include bacterial cells such as Escherichia coli, eukaryotic cells, or he like. In one embodiment, mammalian cells, such as Chinese hamster ovary cells (CHO) can be used.

A variety of host-expression vector systems may be utilized to express the fusion proteins described herein. Such host-expression systems represent vehicles by which the coding sequences of the isolated fusion proteins described herein may be produced and subsequently purified, but also represent cells which may, when transformed or transfected with the appropriate nucleotide coding sequences, express the fusion proteins described herein in situ. These include, but are not limited to, microorganisms such as bacteria (e.g., E. coli and B. subtilis) transformed with recombinant bacteriophage DNA, plasmid DNA or cosmid DNA expression vectors containing coding sequences for the fusion proteins described herein; yeast (e.g., Saccharomyces pichia) transformed with recombinant yeast expression vectors containing sequences encoding the fusion proteins described herein; insect cell systems infected with recombinant virus expression vectors (e.g., baclovirus) containing the sequences encoding the fusion proteins described herein; plant cell systems infected with recombinant virus expression vectors (e.g., cauliflower mosaic virus (CaMV) and tobacco mosaic virus (TMV) or transformed with recombinant plasmid expression vectors (e.g., Ti plasmid) containing sequences encoding the fusion proteins described herein; or mammalian cell systems (e.g., COS, CHO, BHK, 293, 293T, 3T3 cells, lymphotic cells, Per C.6 cells harboring recombinant expression constructs containing promoters derived from the genome of mammalian cells (e.g., metallothionein promoter) or from mammalian viruses (e.g., the adenovirus late promoter; the vaccinia virus 7.5K promoter).

In bacterial systems, a number of expression vectors may be advantageously selected depending upon the use intended for the fusion proteins being expressed. For example, when a large quantity of such a protein is to be produced, for the generation of pharmaceutical compositions of fusion proteins described herein, vectors which direct the expression of high levels of fusion protein products that are readily purified may be desirable. Such vectors include, but are not limited, to the E. coli expression vector pUR278, in which the coding sequence may be ligated individually into the vector in frame with the lac Z coding region so that a fusion protein is produced; pIN vectors, and the like. pGEX vectors may also be used to express foreign polypeptides as fusion proteins with glutathione S-transferase (GST). In general, such fusion proteins are soluble and can easily be purified from lysed cells by adsorption and binding to a matrix glutathione-agarose beads followed by elution in the presence of free glutathione. The pGEX vectors are designed to include thrombin or factor Xa protease cleavage sites so that the cloned target gene product can be released from the GST moiety.

In mammalian host cells, a number of viral-based expression systems may be utilized. In cases where an adenovirus is used as an expression vector, the coding sequence of interest may be ligated to an adenovirus transcription/translation control composition, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing the immunoglobulin molecule in infected hosts. Specific initiation signals may also be required for efficient translation of inserted antibody coding sequences. These signals include the ATG initiation codon and adjacent sequences. Furthermore, the initiation codon must be in phase with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc.

In addition, a host cell strain may be chosen which modulates the expression of the inserted sequences, or modifies and processes the gene product in the specific fashion desired. Such modifications (e.g., glycosylation) and processing (e.g., cleavage) of protein products may be important for the function of the protein. Purification and modification of recombinant proteins is well known in the art such that the design of the polyprotein precursor could include a number of embodiments readily appreciated by a skilled worker. Any known proteases or peptidases known in the art can be used for the described modification of the precursor molecule, e.g., thrombin or factor Xa, enterokinase, furin, and AcTEV, and the Foot and Mouth Disease Virus Protease C3, etc.

Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells which possess the cellular machinery for proper processing of the primary transcript, glycosylation, and phosphorylation of the gene product may be used. Such mammalian host cells include but are not limited to CHO, VERY, BHK, HeLa, COS, MDCK, 293, 293T, 3T3, W138, BT483, Hs578T, HTB2, BT20 and T47D, CRL7030 and Hs578Bst.

For long-term, high-yield production of recombinant proteins, stable expression is preferred. For example, cell lines which stably express fusion proteins described herein may be engineered. Rather than using expression vectors which contain viral origins of replication, host cells can be transformed with DNA controlled by appropriate expression control elements (e.g., promoter, enhancer, sequences, transcription terminators, polyadenylation sites, etc.), and a selectable marker. Following the introduction of the foreign DNA, engineered cells may be allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines. This method may advantageously be used to engineer cell lines which express the fusion proteins described herein. Such engineered cell lines may be particularly useful in screening and evaluation of fusion proteins that interact directly or indirectly with the fusion proteins described herein.

The expression levels of the fusion described herein can be increased by vector amplification. When a marker in the vector system expressing a fusion protein described herein is amplifiable, increase in the level of inhibitor present in culture of host cell will increase the number of copies of the marker gene. Since the amplified region is associated with the nucleotide sequence of a fusion protein described herein or a fusion protein described herein, production of the fusion protein will also increase.

Once a fusion described herein has been recombinantly expressed, it may be purified by any method known in the art for purification of polypeptides, polyproteins or antibodies (e.g., analogous to antibody purification schemes based on antigen selectivity) for example, by chromatography (e.g., ion exchange, affinity, particularly by affinity for the specific antigen (optionally after Protein A selection where the polypeptide comprises an Fc domain (or portion thereof)), and sizing column chromatography), centrifugation, differential solubility, or by any other standard technique for the purification of polypeptides or antibodies.

In some embodiments, fusion proteins may advantageously resist degradation, relative to other compositions comprising connected antibodies and enzymes. Fusion proteins may also result in unexpectedly high performance of the molecule. For example. For example, the enzyme, the antibody, or the antibody fragment may have an unexpectedly high activity when comprised by a fusion protein of a composition, relative to the activity of compositions where the enzyme is cross-linked with the antibody or the antibody fragment. This may advantageously improve the potency of compositions comprising fusion proteins.

However, the enzyme may be connected to another portion of the composition (e.g., an antibody or an antibody portion) via cross-linking. The portion and the enzyme may be crosslinked using a crosslinking agent. The cross-link may comprise a synthetic cross-linker, or may comprise a direct coupling between amino-acid side-chains of the antibody and the enzyme. In some embodiments, the antibody and the enzyme are cross-linked using one or more cross-linking agents. Non-limiting examples of cross-linking agents include glutaraldehyde, NHS-esters (N-hydroxysuccinimide) (e.g., dithiobis(succinimidylpropionate), dithiobis(sulfosuccinimidylpropionate), etc.), PEG groups, imidoesters (e.g., dimethyl adipimidate, dimethyl suberimidate, dimethyl pimelimidate, etc.), maleimides, pyridyls, carbodiimide, isocyanate, or the like.

The antibody and the enzyme may be coupled through any suitable coupling system, e.g., amine-to-amine, sulfhydryl-to-sulfhydryl, amine-to-sulfhydryl, carboxyl-to-amine, sulfhydryl-to-carbohydrate, hydroxyl-to-sulfhydryl, or the like. Those of ordinary skill in the art will be familiar with methods of cross-linking or conjugating proteins to each other.

The composition may include, or may be administered along with, a treatment agent in certain embodiments. The treatment agent may be chosen to increase the susceptibility of cells to the composition. In some embodiments, the subject is pretreated with the treatment agent. In some embodiments, the subject is administered the treatment agent as a part of, or concurrently with the administration of, a composition as described herein. The subject may also receive the treatment agent after administration of the composition and the disclosure is not so limited. Treatment of the subject with the treatment agent may prepare the subject for successful attack on the lysosome membrane, which may be useful for treating the disease. Treatments using a treatment agent may provide for more efficient attack on the lysosome membrane. However, it should also be understood that use of a treatment agent is not required, and in some cases, a composition may be administered without use of a treatment agent.

In some embodiments, the treatment agent is configured to increase a concentration of reactive oxygen species near a cell targeted by the composition. Without wishing to be bound by theory, it is believed that, as discussed above, oxidization processes (e.g., those caused by the action of oxidases or peroxidases) may be useful for treating diseases or conditions. Thus, the treatment agent may increase a rate of oxidation, or may increase a total amount of oxidation, by increasing the concentration of reactive oxygen species near the cell.

The increase in the concentration of reactive oxygen species is, in some embodiments, related to a level of antioxidants in the subject, because antioxidants may reduce or inhibit such tumor treatments. In some embodiments, the treatment agent lowers a level of antioxidants near the targeted cell. For example, the treatment agent may remove or suppress antioxidants of the subject, as discussed in greater detail below.

It should be understood that one or more treatment agents may be used, separately or together, in accordance with some embodiments. For example, to a subject may be administered statins, fatty acids, or both, as discussed in greater detail below. Treatment agents may be administered separately and/or together. If administered separately, treatment agents may be administered simultaneously and/or sequentially, in any suitable order. Any suitable method of administration and dosing schedule may be used, including those discussed herein. Additionally, one or more treatment agents may be present in a suitable, pharmaceutically acceptable carrier. Antioxidants may be removed or suppressed from a subject, with or without the use of a treatment agent.

In some embodiments, the treatment agent comprises a fatty acid. The fatty acid may comprise an unsaturated fatty acid. For example, the treatment agent may comprise an n-3 through n-6 fatty acid. In some embodiments, the treatment agent is the fatty acid. In some embodiments, administration of the fatty acid causes an antioxidant status of the subject to be lowered. Unsaturated fatty acids, including n-3 through n-6 fatty acids, may be fed to the subject, or may be administered by any of a variety of other appropriate methods. In the context of the present disclosure, it has been inventively recognized that treating a subject with unsaturated fatty acids may increase a local concentration of unsaturated fatty acids that reactive oxygen species can oxidize. This may accelerate lysosome-induced cell-death, by accelerating degradation of lysosomal membranes.

One or more fatty acids may be administered to a subject. The one or more fatty acids may be administered simultaneously and/or sequentially. The unsaturated fatty acid may include one or more double bonds and/or triple bonds within the fatty acid chain. The fatty acid may be a relatively small-chain fatty acids. Non-limiting examples of such unsaturated fatty acids include n-3 through n-8 (e.g., n-3, n-4, n-5, n-6, n-7, or n-8) fatty acids. Unsaturated fatty acids may be monounsaturated and/or polyunsaturated fatty acids. Non-limiting examples of unsaturated fatty acids include, but are not limited to, CH₂=CHCOOH, CH₃CH═CHCOOH, CH₃CH₂CH═CHCOOH, CH₃CH₂CH₂CH═CHCOOH, CH₂=CHCH₂COOH, CH₂=CH CH₂CH₂COOH, CH₂=CHCH₂CH₂CH₂COOH, CH₂=CHCH₂CH═CHCOOH, CH₂=CHCH═CHCOOH, etc. In addition, in some cases, the fatty acids may come from naturally occurring sources, such as krill oil, fish oil, safflower oil, soybean oil, flaxseed oil, canola oil, algal oil, etc.

A wide range of dosing of fatty acids may be used. In some embodiments, fatty acids may be given to a subject, such as a human, at a dosage of at least 0.1 g, at least 0.2 g, at least 0.3 g, at least 0.4 g, at least 0.5 g, at least 0.6 g, at least 0.7 g, at least 0.8 g, at least 0.9 g, at least 1 g, at least 2 g, at least 3 g, at least 4 g, at least 5 g, at least 6 g, at least 7 g, at least 8 g, at least 9 g, at least 10 g, at least 11 g, at least 12 g, at least 13 g, at least 14 g, or at least 15 g. In some embodiments, the fatty acids may be given at a dosage of no more than 15 g, no more than 14 g, no more than 13 g, no more than 12 g, no more than 11 g, no more than 10 g, no more than 9 g, no more than 8 g, no more than 7 g, no more than 6 g, no more than 5 g, no more than 4 g, no more than 3 g, no more than 2 g, no more than 1 g, no more than 0.9 g, no more than 0.8 g, no more than 0.7 g, no more than 0.6 g, no more than 0.5 g, no more than 0.4 g, no more than 0.3 g, no more than 0.2 g, or no more than 0.1 g. Combinations of any of these are also possible in some embodiments, e.g., the dosage may be between 1 and 15 g, between 1 and 10 g, between 5 and 10 g, between 0.5 and 1 g, etc.

In some embodiments, the fatty acids may be given to a subject, such as a human, at a dosage of at least 1 mg/kg, at least 2 mg/kg, at least 3 mg/kg, at least 5 mg/kg, at least 10 mg/kg, at least 20 mg/kg, at least 30 mg/kg, at least 50 mg/kg, at least 100 mg/kg, at least 200 mg/kg, at least 300 mg/kg, at least 500 mg/kg, at least 1 g/kg, or at least 2 mg/kg. In some cases, the fatty acids may be given at a dosage of no more than 2 g/kg, no more than 1 g/kg, no more than 500 mg/kg, no more than 300 mg/kg, no more than 200 mg/kg, no more than 100 mg/kg, no more than 50 mg/kg, no more than 30 mg/kg, no more than 20 mg/kg, no more than 10 mg/kg, no more than 5 mg/kg, no more than 3 mg/kg, no more than 2 mg/kg, or no more than 1 mg/kg. Combinations of any of these are also possible in some embodiments, e.g., the dosage may be 1 to 2 g/kg, 500 mg/kg to 1 g/kg, 400 to 800 mg/kg, etc. Appropriate doses, dose rates, and treatments using the fatty acids may be determined using the methods described herein.

In some embodiments, the treatment agent comprises a statin. The statin may be co-administered with fatty acids, or may be administered without administration of fatty acids. Statins, also known as HMG-CoA reductase inhibitors, may be used to lower lipid levels. Non-limiting examples of statins include atorvastatin, fluvastatin, lovastatin, pitavastatin, pravastatin, rosuvastatin, cerivastatin, mevastatin, and simvastatin. One or more statins may be administered to a subject, e.g., simultaneously and/or sequentially.

A wide range of dosing of statins may be used. For example, the statins may be given to a subject, such as a human, at a dosage of at least 1 mg, at least 2 mg, at least 3 mg, at least 5 mg, at least 10 mg, at least 20 mg, at least 30 mg, at least 40 mg, at least 50 mg, at least 60 mg, at least 70 mg, at least 80 mg, at least 90 mg, or at least 100 mg. In some cases, the statins may be given at a dosage of no more than 100 mg, no more than 90 mg, no more than 80 mg, no more than 70 mg, no more than 60 mg, no more than 50 mg, no more than 40 mg, no more than 30 mg, no more than 20 mg, no more than 10 mg, no more than 5 mg, no more than 3 mg, no more than 2 mg, or no more than 1 mg. Combinations of any of these are also possible in some embodiments, e.g., the dosage may be 10 to 20 mg, 20 to 40 mg, 40 to 80 mg, 5 to 10 mg, etc.

In addition, in some embodiments, statins may be given to a subject, such as a human, at a dosage of at least 1 mg/kg, at least 2 mg/kg, at least 3 mg/kg, at least 4 mg/kg, at least 5 mg/kg, at least 6 mg/kg, at least 7 mg/kg, at least 8 mg/kg, at least 9 mg/kg, at least 10 mg/kg, at least 11 mg/kg, at least 12 mg/kg, at least 13 mg/kg, at least 14 mg/kg, or at least 15 mg/kg. In some embodiments, the statins may be given at a dosage of no more than 15 mg/kg, no more than 14 mg/kg, no more than 13 mg/kg, no more than 12 mg/kg, no more than 11 mg/kg, no more than 10 mg/kg, no more than 9 mg/kg, no more than 8 mg/kg, no more than 7 mg/kg, no more than 6 mg/kg, no more than 5 mg/kg, no more than 4 mg/kg, no more than 3 mg/kg, no more than 2 mg/kg, or no more than 1 mg/kg. Combinations of any of these are also possible in some embodiments, e.g., the dosage may be between 1 and 15 mg/kg, between 1 and 10 mg/kg, between 5 and 10 mg/kg, etc. Appropriate doses, dose rates, and treatments using the fatty acids may be determined using the methods described herein.

According to certain embodiments, if a subject is on antioxidants, the antioxidants may be removed or suppressed from the subject. For example, the antioxidants may be withheld entirely, or the antioxidant dosage may be reduced, e.g., in amount and/or frequency, as part of preparing the tumor for treatment. Non-limiting examples of antioxidants include beta carotene, vitamin A, and vitamin E. Other examples include vitamin C, glutathione, lipoic acid, uric acid, and ubiquinol.

As mentioned, some embodiments of the present disclosure are directed towards a composition comprising an enzyme, such as an oxidoreductase. For example, the enzyme may be oxidase or a peroxidase. Other exemplary oxidoreductases that may be used include, but are not limited to transglutaminase 3 (e.g., human transglutaminase 3); hydroxyacid oxidase 1 (e.g., human hydroxyacid oxidase 1); nitrate reductase (NAD[P]H) (e.g., from Pichia pastoris); native Aspergillus niger catalase; NADH peroxidase (e.g., from Pseudomona flurescens); L-malate dehydrogenase (e.g., from E. coli); L-lactate dehydrogenase (e.g., porcine L-lactate dehydrogenase); D-lactate dehydrogenase (e.g., from Leuconostoc mesenteroides); isocitrate dehydrogenase (e.g., isocitrate dehydrogenase from Bacillus subtilis); myo-Inositol dehydrogenase (e.g., from Bacillus subtilis); prokaryotic 3-hydroxybutyrate dehydrogenase; galactose dehydrogenase (e.g., from E. Coli); galactose mutarotase (e.g., from E. Coli); formate dehydrogenase (e.g., from Candida boidinii); manganese peroxidase (e.g., native Nematoloma frowardii manganese peroxidase); cholesterol oxidase (e.g., from Nocardia sp.); 15-lipoxygenase-2 (e.g., human 15-lipoxygenase-2); 3-Acetylpyridine-Adenine Dinucleotide phosphate; 3-oxo-5-β-steroid 4-dehydrogenase (e.g., human 3-oxo-5-β-steroid 4-dehydrogenase); 3α-hydroxysteroid dehydrogenase; 5-lipoxygenase (e.g., human 5-lipoxygenase); acyl-CoA oxidase; acyl-coenzyme A dehydrogenase 8 (e.g., human acyl-coenzyme A dehydrogenase 8); aflatoxin B1 aldehyde reductase member 2 (e.g., human aflatoxin B1 aldehyde reductase member 2); alanine dehydrogenase (e.g., from Bacillus cereus); alcohol dehydrogenase (e.g., human alcohol dehydrogenase or alcohol dehydrogenase from E. coli); Aldehyde dehydrogenase 2 (e.g., human aldehyde dehydrogenase 2); Aldose reductase (e.g., human aldose reductase); Bilirubin oxidase; Biotinylated Luciferase (e.g., from E. coli); bovine catalase-polyethylene glycol; bovine superoxide dismutase-polyethylene glycol; catalase (e.g., from psychrotolerant bacteria); cholesterol oxidase (e.g., from E. coli); Cu/Zn superoxide dismutase; cyclohexanone monooxygenase (e.g., from Acinetobacter sp.); cyclooxygenase (e.g., human cyclooxygenase 1 or 2, or ovine cyclooxygenase 2); cytochrome p450 reductase (e.g., human cytochrome p450 reductase); D-2-hdroxyglutarate dehydrogenase (e.g., from Acidaminococcus fermentans); D-2-hdroxyisocaproic aid dehydrogenase; D-3-hydroxybutyrate dehydrogenase; D-amino acid dehydrogenase; D-amino acid oxidase (e.g., human D-amino acid oxidase); diaphorase (e.g., from E. coli); fhydrofolate reductase (e.g., human dihydrofolate reductase); fimethylglycine oxidase (e.g., from Arthrobacter globifomis); Disulfide oxidoreductase; D-lactate dehydrogenase (e.g., from E. coli); DT diaphorase (e.g., human DT diaphorase or rat DT diaphorase); FMN reductase (e.g., from E. coli); formate dehydrogenase (e.g., from Candida boidinii, or recombinant E. coli); free methionine-(R)-sulfoxide reductase (e.g., from E. coli); fructosyl-amino acid oxidase (e.g., from Corynebacterium sp. or E. coli); fructosyl-amino acid oxidase; fructosyl-peptide oxidase (e.g., from E. coli); galactose dehydrogenase (e.g., from recombinant E. coli); galactose oxidase (e.g., from Dactylium dendroides); glucose dehydrogenase (e.g., from E. coli); glucose-6-phosphate dehydrogenase (e.g., from E. coli); glutamate dehydrogenase (e.g., from E. coli or from thermophilic bacterium); glutathione reductase (e.g., human glutathione reductase from E. coli); glyceraldehyde-3-phosphate dehydrogenase (e.g., human or mouse glyceraldehyde-3-phosphate dehydrogenase); glycerol-3-phosphate dehydrogenase (e.g., from E. coli); glycerol-3-phosphate oxidase (e.g., from E. coli); Glycine oxidase H244K (e.g., from Bacillus subtilis); histamine dehydrogenase (e.g., from E. coli); inosine monophosphate dehydrogenase (e.g., from Staphylococcus aureus); inosine monophosphate dehydrogenase Type II (e.g., human inosine monophosphate dehydrogenase Type II); isocitrate dehydrogenase (NAD+) (e.g., from bacteria or yeast); ketoamine oxidase; ketoreductase; L-2-hydroxyisocaproic acid dehydrogenase; laccase (e.g., from Bacillus subtilis or E. coli); lactaldehyde dehydrogenase (e.g., from E. coli); lactate dehydrogenase (e.g., native human lactate dehydrogenase 1, 2, 3, or 5, or chicken heart lactate dehydrogenase); lactate oxidase (e.g., from Aerococcus viridans); lactic dehydrogenase; leucine dehydrogenase; Leuconostoc mesenteroides glucose-6-phosphate dehydrogenase; L-glutamate dehydrogenase; L-glutamate oxidase (e.g., from Streptomyces sp.); L-lactate dehydrogenase; L-lactic dehydrogenase (e.g., from Bacillus stearothermophilus); L-leucine dehydrogenase; L-phenylalanine dehydrogenase; luciferase (e.g., from E. coli or from Photinus pyralis); lytic cellulose monooxygenase (e.g., from Thermobifida fusca); lytic chitin monooxygenase (e.g., from Bacillus licheniformis or from Lactococcus lactis); malate dehydrogenase; mannitol dehydrogenase; Mn-superoxide dismutase; monoamine oxidase (e.g., human monoamine oxidase A or human monoamine oxidase B); isocitrate dehydrogenase 1 R132H (e.g., human isocitrate dehydrogenase 1 R132H or isocitrate dehydrogenase 1 R132H from E. coli); N-Acylmannosamine 1-dehydrogenase (e.g., from Pseudomonas sp.); native 6-phospho-D-gluconate dehydrogenase (e.g., from E. coli); native Acremonium sp. ascorbate oxidase; native Aerococcus viridans glycerol 3-phosphate oxidase; native Aerococcus viridans pyruvate oxidase; native Agaricus bisporus laccase; native Alcaligenes faecalis 3-hydroxybutyrate dehydrogenase; native Alcaligenes sp. choline oxidase; native alcohol dehydrogenase; native Arthrobacter globiformis choline oxidase; native Arthrobacter globiformis uricase; native Arthrobacter sp. acyl-CoA oxidase; native Arthrobacter sp. tyramine oxidase; native Aspergillus niger glucose oxidase; native Aspergillus niger nitrate reductase (NAD[P]H); native Aspergillus sp. catalase; native Aspergillus sp. glucose oxidase; native Bacillus cereus L-leucine dehydrogenase; native Bacillus fastidiosus uricase; native Bacillus licheniformis NADH oxidase; native Bacillus megaterium diaphorase (NADH); native Bacillus pumilus bilirubin oxidase/Laccase; native Bacillus sp. glucose-6-phosphate dehydrogenase; native Bacillus sp. leucine dehydrogenase; native Bacillus sp. sarcosine oxidase; native Bacillus sp. uricase; native Bacillus sp. 12α-hydroxysteroid dehydrogenase; native Bacillus stearothermophilus alanine dehydrogenase; native Bacillus stearothermophilus diaphorase 1; native Bacillus stearothermophilus leucine dehydrogenase; native Bacillus stearothermophilus polynucleotide phosphorylase; native Bacillus stearothermophilus superoxide dismutase; native Bacillus subtilis bilirubin oxidase; native Bacillus subtilis L-alanine dehydrogenase; native baker's yeast (S. cerevisiae) glucose-6-phosphate dehydrogenase; native baker's yeast (S. cerevisiae) glutathione reductase; native baker's yeast (S. cerevisiae) glyceraldehyde-3-phosphate dehydrogenase; native Bjerkandera adusta peroxidase; native bovine catalase; native bovine glutamate dehydrogenase; native bovine glutathione peroxidase; native bovine lactoperoxidase; native bovine L-glutamic dehydrogenase; native bovine L-lactic dehydrogenase; native bovine L-lactic dehydrogenase; native bovine malic dehydrogenase; native bovine plasma amine oxidase; native bovine superoxide dismutase; native bovine xanthine oxidase; native Caldariomyces fumago chloroperoxidase; native Candida boidinii formate dehydrogenase; native Candida sp. alcohol oxidase; native Candida sp. uricase; native Candida utilis L-glutamic dehydrogenase (NADP); native canine superoxide dismutase; native Cellulomonas sp. glycerol dehydrogenase; native chicken glyceraldehyde-3-phosphate dehydrogenase; native chicken L-lactic dehydrogenase; native chicken malic dehydrogenase (oxaloacetate-decarboxylating); native chicken sulfite oxidase; native Clostridium kluyveri diaphorase; native Clostridium sp. diaphorase; native Corallina officinalis bromoperoxidase; native Coriolus versicolor laccase; native corn nitrate reductase; native Corynebacterium glutamicum catalase; native Crotalus adamanteus L-Amino Acid oxidase; native Crotalus atrox L-amino acid oxidase; native Crotalus durissus venom L-Amino Acid oxidase; native Cucurbita sp. ascorbate oxidase; native Cucurbita sp. L-ascorbate oxidase; native Dactylium dendroides galactose oxidase; native diaphorase (NADPH) (e.g., from Bacillus megaterium); native E. coli 1-5-anhydroglucitol-6-phosphate dehydrogenase; native E. coli galactose 1-dehydrogenase; native E. coli sarcosine oxidase; native Enterobacter aerogenes glycerol dehydrogenase; native Enterobacter aerogenes myo-Inositol dehydrogenase; native equine spleen apoferritin; native E. coli nitrate reductase (cytochrome); native E. coli superoxide dismutase; native E. coli thioredoxin reductase; native Gluconobacter industrius D-fructose dehydrogenase; native Gluconobacter sp. D-fructose dehydrogenase; native glutamate dehydrogenase (NADP+); native glycine max (soybean) lipoxidase; native Hansenula sp. alcohol oxidase; native horseradish apoperoxidase; native horseradish peroxidase; native horseradish poly peroxidase; native horseradish superoxide dismutase; native human catalase; native human eosinophil peroxidase; native human glutathione peroxidase; native human glyceraldehyde-3-phosphate dehydrogenase; native human lactate dehydrogenase; native human lactoperoxidase; native human myeloperoxidase (e.g. isoform A, B, or C); native human superoxide dismutase; native laccase (e.g., from Cerrena unicolor or from white rot fungi); native lactate dehydrogenase (e.g., from Thermophillic bacteria); native Lactobacillus delbrückii D-lactate dehydrogenase; native Lactobacillus leichmanii D-lactic dehydrogenase; native Leuconostoc mesenteroides D-lactic dehydrogenase; native Leuconostoc mesenteroides glucose-6-phosphate dehydrogenase; native Leuconostoc mesenteroides Mannitol dehydrogenase; native lignin peroxidase (e.g., from Phanerochaete chrysosporium); native malate dehydrogenase (e.g., from thermophillic bacteria); native Micrococcus lysodeikticus catalase; native microorganism cholesterol oxidase; native microorganism D-lactate dehydrogenase; native microorganism glucose dehydrogenase (FAD-dependent); native microorganism glucose dehydrogenase (NAD(P)-dependent); native microorganism glucose dehydrogenase (PQQ-dependent); native microorganism glucose-6-phosphate dehydrogenase; native microorganism glutamate dehydrogenase (NAD-dependent); native microorganism L-α-glycerophosphate oxidase; native microorganism malate dehydrogenase; native microorganism P-hydroxybenzoate hydroxylase; native microorganism pyruvate oxidase; native microorganism sarcosine oxidase; native microorganism sorbitol dehydrogenase; native microorganism xanthine oxidase; native microorganisms pyruvate oxidase; native murine catalase; native mushroom tyrosinase; native mushrooms polyphenol oxidase; native Myrothecium verrucaria bilirubin oxidase; native nitrate reductase (e.g., from Aspergillus species); native Nocardia erythropolis cholesterol oxidase; native Nocardia sp. cholesterol dehydrogenase; native Pediococcus sp. glycerol 3-phosphate oxidase; native Pediococcus sp. glycerol-3-phosphate oxidase; native Pediococcus sp. lactate oxidase; native Pediococcus sp. L-α-glycerophosphate oxidase; native Photinus pyralis (firefly) luciferase; native Photobacterium phosphoreum (Lux) bacterial luciferase; native Pichia pastoris alcohol oxidase; native plant origin diamine oxidase; native Pleurotus ostreatus laccase; native porcine apo D-amino acid oxidase; native porcine cytochrome C reductase; native porcine D-amino acid oxidase; native porcine diamine oxidase; native porcine heart lactate dehydrogenase; native porcine isocitric dehydrogenase (NADP); native porcine lactate dehydrogenase; native porcine lipoamide dehydrogenase; native porcine L-lactate dehydrogenase; native porcine L-lactic dehydrogenase; native porcine malate dehydrogenase, IFCC Qualityporcine heart; native porcine malic dehydrogenase; native porcine NAD(P)H dehydrogenase (quinone); native porcine superoxide dismutase; native porcine α-ketoglutarate dehydrogenase; native Proteus sp. glutamate dehydrogenase (NADP-dependent); native Proteus sp. L-glutamic dehydrogenase (NADP); native Pseudomonas fluorescens galactose 1-dehydrogenase; native Pseudomonas lemoignei β-hydroxybutyrate dehydrogenase; native Pseudomonas sp. cholesterol oxidase; native Pseudomonas sp. D-3-hydroxybutyrate dehydrogenase; native Pseudomonas sp. Formaldehyde dehydrogenase; native Pseudomonas sp. glucose dehydrogenase.; native Pseudomonas sp. N-acylhexosamine oxidase; native Pseudomonas sp. p-hydroxybenzoate hydroxylase; native Pseudomonas sp. Protocatechuate 3, 4-dioxygenase; native Pseudomonas sp. Protocatechuate 3,4-Dioxygenase; native Pseudomonas sp. Sarcosine dehydrogenase; native Pseudomonas testosteroni 3α-hydroxysteroid dehydrogenase; native rabbit cytochrome P450 reductase; native rabbit glyceraldehyde-3-phosphate dehydrogenase; native rabbit glycerol-3-phosphate dehydrogenase; native rabbit lactate dehydrogenase; native rabbit L-lactic dehydrogenase; native rabbit pyruvate kinase/lactic dehydrogenase enzymes rabbit muscle; native rabbit α-glycerophosphate dehydrogenase; native rabbit α-glycerophosphate dehydrogenase-triosephosphate isomerase; native rat sorbitol dehydrogenase; native rat thioredoxin reductase; native Rhodopseudomonas sphaeroides β-hydroxybutyrate dehydrogenase; native Rhus vernicifera laccase; native roxburgh superoxide dismutase; native Saccharomyces cerevisiae alcohol dehydrogenase; native sheep 6-phosphogluconic dehydrogenase; native sheep cyclooxygenase; native sheep sorbitol dehydrogenase; native spinach glutathione reductase; native Spinacia oleracea (spinach) ferredoxin-NADP+ reductase; native Sporosarcina sp. L-phenylalanine dehydrogenase; native Staphylococcus epidermidis D-lactic dehydrogenase; native Streptococcus thermophilus glycerol 3-phosphate oxidase; native Streptomyces sp. cholesterol oxidase; native Thermoactinomyces intermedius phenylalanine dehydrogenase; native Thermoanaerobium brockii alcohol dehydrogenase, NADP+ dependent; native Thermoanaerobium sp. aromatic alcohol dehydrogenase, NADP+ dependent; native thermostable fungi catalase for semiconductor process; native thermostable fungi catalase for textile process; native Thermus flavus malic dehydrogenase; native torula yeast glucose-6-phosphate dehydrogenase; native Trametes versicolor laccase; native Trichoderma viride lysine oxidase; native versatile peroxidase (e.g., from Bjerkandera adusta); native vibrio fischeri (Photobacterium Luciferase); native wheat germ glutathione reductase; native white-rot fungus (Phaner ochaete chrysosporium) manganese peroxidase; native xanthine dehydrogenase (e.g., from bovine milk); native xanthine dehydrogenase; native yeast 6-phosphogluconic dehydrogenase; native yeast alcohol dehydrogenase; native yeast aldehyde dehydrogenase; native yeast formate dehydrogenase; native yeast malate dehydrogenase; native zucchini ascorbate oxidase; native Zymomonas mobilis alcohol dehydrogenase; native Zymomonas mobilis glucokinase; native Zymomonas mobilis glucose-6-phosphate dehydrogenase; NiFe-type cytoplasmic hydrogenase (e.g., from Pyrococcus furiosus); nitrate reductase (e.g., from Arabidopsis thaliana); nitric oxide synthase (e.g., mouse nitric oxide synthase); nitroreductase (e.g., from E. coli); oxalate oxidase (e.g., from B. subtilis); phosphite dehydrogenase; phosphogluconate dehydrogenase (e.g., human phosphogluconate dehydrogenase or phosphogluconate dehydrogenase from E. coli); prokaryotic galactose dehydrogenase; prostaglandin F synthase (e.g., human prostaglandin F synthase); protocatechuate 3,4-dioxygenase; pyranose oxidase (e.g., from Coriolus sp. or from E. coli); pyruvate oxidase (e.g., from E. coli); R-carbonyl reductase; recombinant ketol-acid reductoisomerase (e.g., from Mycobacterium tuberculosis); sarcosine oxidase (e.g., from E. coli); S-carbonyl reductase; secondary alcohol dehydrogenase; sulfite oxidase (e.g., human sulfite oxidase); taurine dioxygenase (e.g., from E. coli); thioredoxin reductase (NADPH) (e.g., from yeast); thyroid peroxidase (e.g., human thyroid peroxidase); UDP-Glc dehydrogenase (e.g., from Streptococcus pyogenes); uricase (e.g., from Candida utilis or from E. coli); uronate dehydrogenase (e.g., from Agrobacterium tumefaciens); valine dehydrogenase; xanthine oxidase (e.g., from Arthrobacter); α-hydroxysteroid dehydrogenase (e.g., from B. choshinensis); or β-galactose dehydrogenase S (e.g., from Pseudomonas fluorescens).

The compositions described herein may be administered to the subject (e.g., a human) using any of a variety of suitable techniques, as discussed in greater detail below. The composition may be administered to the subject in any suitable dose. For example, the composition may be administered in a dosage of greater than or equal to 10 mg, greater than or equal to 15 mg, greater than or equal to 20 mg, greater than or equal to 25 mg, greater than or equal to 30 mg, greater than or equal to 40 mg, greater than or equal to 50 mg, greater than or equal to 60 mg, greater than or equal to 70 mg, greater than or equal to 80 mg, greater than or equal to 90 mg, greater than or equal to 100 mg, greater than or equal to 200 mg, greater than or equal to 300 mg, greater than or equal to 500 mg, greater than or equal to 1000 mg, greater than or equal to 1500 mg, greater than or equal to 2000 mg, greater than or equal to 2500 mg, greater than or equal to 3000 mg, greater than or equal to 5000 mg, etc. In some embodiments, the composition may be applied at a dosage of less than or equal to 5000 mg, less than or equal to 3000 mg, less than or equal to 2500 mg, less than or equal to 2000 mg, less than or equal to 1500 mg, less than or equal to 1000 mg, less than or equal to 500 mg, less than or equal to 300 mg, less than or equal to 200 mg, less than or equal to 100 mg, less than or equal to 90 mg, less than or equal to 80 mg, less than or equal to 70 mg, less than or equal to 60 mg, less than or equal to 50 mg, less than or equal to 40 mg, less than or equal to 30 mg, less than or equal to 25 mg, less than or equal to 20 mg, less than or equal to 15 mg, less than or equal to 10 mg, etc. Combinations of these ranges are also possible. For example, the dosage may be greater than or equal to 10 mg and less than or equal to 20 mg, greater than or equal to 50 mg and less than or equal to 100 mg, or greater than or equal to 100 mg and less than or equal to 200 mg.

In some embodiments, the compositions may be given to a subject (e.g., a human) at a dosage of greater than or equal to 1 mg/kg, greater than or equal to 2 mg/kg, greater than or equal to 3 mg/kg, greater than or equal to 4 mg/kg, greater than or equal to 5 mg/kg, greater than or equal to 6 mg/kg, greater than or equal to 7 mg/kg, greater than or equal to 8 mg/kg, greater than or equal to 9 mg/kg, greater than or equal to 10 mg/kg, greater than or equal to 11 mg/kg, greater than or equal to 12 mg/kg, greater than or equal to 13 mg/kg, greater than or equal to 14 mg/kg, or greater than or equal to 15 mg/kg. In some embodiments, the statins maybe given at a dosage of less than or equal to 15 mg/kg, less than or equal to 14 mg/kg, less than or equal to 13 mg/kg, less than or equal to 12 mg/kg, less than or equal to 11 mg/kg, less than or equal to 10 mg/kg, less than or equal to 9 mg/kg, less than or equal to 8 mg/kg, less than or equal to 7 mg/kg, less than or equal to 6 mg/kg, less than or equal to 5 mg/kg, less than or equal to 4 mg/kg, less than or equal to 3 mg/kg, less than or equal to 2 mg/kg, or less than or equal to 1 mg/kg. Combinations of these ranges are also possible. For example, in some embodiments, the dosage is greater than or equal to 1 mg/kg and less than or equal to 15 mg/kg, greater than or equal to 1 mg/kg and less than or equal to 10 mg/kg, greater than or equal to 5 mg/kg or less than or equal to 10 mg/kg.

In another aspect, a composition as described herein may be administered to a subject. The composition may be administered by itself. In some embodiments, the composition is administered in conjunction with co-factors, other therapeutics (e.g., treatment agents), or the like. For example, a composition may be administered alone, or in conjunction with substrates such as hypoxanthine, xanthine, glucose, or the like, e.g., as discussed herein, and/or in conjunction with fatty acids, statins, etc. See, for example, Int. Pat. Apl. Pub. No. WO 2019/099687, incorporated herein by reference.

The compositions may be applied in a therapeutically effective, pharmaceutically acceptable amount as a pharmaceutically acceptable formulation, for example, a pharmaceutically acceptable carrier such as those described below. The term “effective amount” of a composition, such as the compositions described herein, refers to the amount necessary or sufficient to realize a desired biologic effect. For example, an effective amount of a composition to treat a tumor may be an amount sufficient to reduce the tumor's size. Combined with the teachings provided herein, by choosing among the various active compositions and weighing factors such as potency, relative bioavailability, subject body weight, severity of adverse side effects and mode of administration, an effective prophylactic or therapeutic treatment regimen can be planned which does not cause substantial toxicity and yet is entirely effective to treat the particular subject. The effective amount for any particular application can vary depending on such factors as the disease or condition being treated, the particular compositions being administered the size of the subject, or the severity of the disease or condition. One of ordinary skill in the art can empirically determine the effective amount of the compositions without necessitating undue experimentation.

The terms “treat,” “treated,” “treating,” and the like, when used herein, refer to administration of the compositions to a subject which may increase the resistance of the subject to a disease, or to further progression of the disease, to control progression of the disease, and/or slow the progression of or to reduce the severity of symptoms of the disease. The effective amount may depend on the particular disease being treated and the desired outcome.

In some embodiments, a therapeutically effective dose of a composition can be initially determined from an animal model. The applied dose may be adjusted based on the relative bioavailability and potency of the administered compound. Adjusting the dose to achieve maximal efficacy based on the methods described above and other methods as are well-known in the art is well within the capabilities of the ordinarily skilled artisan.

In administering the composition to a subject, dosing amounts, dosing schedules, routes of administration, and the like may be selected so as to affect known activities of these compositions. Dosages may be estimated based on the results of experimental models, optionally in combination with the results of assays of compositions described herein. Dosage may be adjusted appropriately to achieve desired drug levels, local or systemic, depending upon the mode of administration. The doses may be given in one or several administrations per day. Multiple doses per day may be used to achieve appropriate systemic levels of the compositions within the subject or within the cell targeted by the composition.

The dose may be chosen to provide a therapeutically effective amount of the composition to the cell targeted by the composition. The dosage may be given in a maximum safe dose. The maximum safe dose may be chosen such that a high therapeutically effective amount of the composition is delivered to the subject but such that a risk to the subject of potentially detrimental side effects is minimal.

The dose of the composition may be chosen to have a desired concentration at the targeted cell, to have a desired efficacy, to have a desired longevity within the subject, to have a desired rate of administration, to have a desired frequency of administration, to act in an appropriate fashion when administered concurrently with other treatments (e.g., as in a cocktail), or for any of a variety of other purposes known to those of ordinary skill in the art.

The dose may be chosen based, at least in part, on conditions associated with the subject. For example, the dose may be chosen based at least in part on the species, age, sex, weight, size, environment, metabolism, physical condition, or current state of health of the subject. In some cases, a subject-specific maximum dose may be used. In some cases, the dose may be administered in a way that limits deleterious effects to the subject (e.g., the dose may be delivered orally, nasally, intravenously, etc., depending on the needs of the subject).

Doses of the composition may be administered daily, weekly, or monthly and any other amount of time therebetween.

A dose may include the composition in any appropriate amount. In some embodiments, the dose of the composition is greater than or equal to 0.1 micrograms, greater than or equal to 0.5 micrograms, greater than or equal to 10 micrograms, greater than or equal to 50 micrograms, greater than or equal to 100 micrograms, greater than or equal to 500 micrograms, greater than or equal to 1000 micrograms, or more. In some embodiments, the dose of the composition is less than or equal to 10000 micrograms, less than or equal to 5000 micrograms, less than or equal to 1000 micrograms, less than or equal to 500 micrograms, less than or equal to 100 micrograms, less than or equal to 50 micrograms, or less. Combinations of these ranges are also possible. For example, in some embodiments the dose of the composition is greater than or equal to 0.5 micrograms and less than or equal to 10000 micrograms.

Doses of the composition may be administered with any of a variety of average rates. In some embodiments, the doses are administered at a rate of greater than or equal to 0.01 micrograms/hour, greater than or equal to 0.05 micrograms/hour, greater than or equal to 0.1 micrograms/hour, greater than or equal to 0.5 micrograms/hour, greater than or equal to 1 micrograms/hour, greater than or equal to 5 micrograms/hour, greater than or equal to 10 micrograms/hour, greater than or equal to 50 micrograms/hour, greater than or equal to 100 micrograms/hour, greater than or equal to 500 micrograms/hour, greater than or equal to 1000 micrograms/hour, or greater. In some embodiments, the doses are administered at a rate of less than or equal to 10000 micrograms/hour, less than or equal to 5000 micrograms/hour, less than or equal to 1000 micrograms/hour, less than or equal to 500 micrograms/hour, less than or equal to 100 micrograms/hour, less than or equal to 50 micrograms/hour, less than or equal to 10 micrograms/hour, less than or equal to 1 micrograms/hour, less than or equal to 0.5 micrograms/hour, less than or equal to 0.1 micrograms/hour, or less. Combinations of these ranges are possible. For example, in some embodiments, the doses are administered at a rate of greater than or equal to 0.01 micrograms/hour, and less than or equal to 10000 micrograms/hour.

The composition may be administered over extended period of time. In some embodiments, the dose is administered over a period of greater than or equal to 1 h, greater than or equal to 2 h, greater than or equal to 4 h, greater than or equal to 6 h, greater than or equal to 12 h, greater than or equal to 24 h, greater than or equal to 3 days, greater than or equal to 7 days, greater than or equal to 10 days, greater than or equal to 14 days, greater than or equal to 21 days, greater than or equal to 30 days, greater than or equal to 45 days, greater than or equal to 60 days, greater than or equal to 90 days, or greater. In some embodiments, the dose is administered over a period of less than or equal to 365 days, less than or equal to 200 days, less than or equal to 90 days, less than or equal to 60 days, less than or equal to 45 days, less than or equal to 30 days, less than or equal to 21 days, less than or equal to 14 days, less than or equal to 10 days, less than or equal to 7 days, less than or equal to 3 days, less than or equal to 24 h, less than or equal to 12 h, or less. Combinations of these ranges are possible. For example, in some embodiments, the dose is administered over a period of greater than or equal to 1 h and less than or equal to 365 days.

The subject may be any appropriate subject. For example the subject may be a mammal, such as a human, or a non-human animal, such as a dog, a cat, a horse, a rabbit, a cow, a pig, a sheep, a goat, a rat (e.g., Rattus Norvegicus), a mouse (e.g., Mus musculus), a guineapig, a non-human primate (e.g., a monkey, a chimpanzee, a baboon, an ape, a gorilla, etc.), or the like.

Administration of a composition of the disclosure may be accomplished by any of a variety of medically acceptable methods that allows the composition to reach its target cell. The particular mode selected may depend of course, upon factors such as those previously described, for example, the particular composition, the severity of the state of the subject being treated, the dosage required for therapeutic efficacy, etc. As used herein, a “medically acceptable” mode of treatment is a mode able to produce effective levels of the compositions within the subject without causing clinically unacceptable adverse effects. In the case of neurodegenerative diseases the blood brain barrier may prevent the agent from reaching the senescent cells. In this case the medically acceptable mode may be administration through the cerebral spinal fluid, i.e., intrathecally.

Any medically of a variety of acceptable methods may be used to administer the compositions to the subject. The administration may be localized (e.g., to a particular region, physiological system, tissue, organ, or cell type) or systemic, depending on the condition to be treated. For example, the compositions may be administered orally, vaginally, rectally, buccally, pulmonary, topically, nasally, transdermally, through parenteral injection or implantation, via surgical administration, or any other method of administration where access to the tumor is achieved. In some cases, more than one method of administration may be used, e.g., if two or more compositions are to be administered.

Examples of parenteral methods of administration that can be used include intravenous, intradermal, subcutaneous, intracavity, intramuscular, intraperitoneal, epidural, or intrathecal administration. Examples of implantation methods of administration include implantation or injection of any implantable or injectable drug delivery system. Oral administration may be used in some embodiments. Oral administration may be advantageous because of the convenience to the subject as well as the dosing schedule. A composition suitable for oral administration may be administered using discrete units, such as hard or soft capsules, pills, cachettes, tablets, troches, dissolving films or lozenges. Other oral compositions suitable for use include solutions or suspensions such as a syrup, an elixir, or an emulsion. The solutions or suspensions may comprise aqueous or non-aqueous liquids. In some embodiments, a composition may be used to fortify a food or a beverage.

The compositions, when it is desirable to deliver them systemically, may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions, or emulsions in oily or aqueous vehicles, and may contain formulatory agents such as suspending, stabilizing and/or dispersing agents.

Pharmaceutical formulations for parenteral administration may include aqueous solutions of the active compounds in water soluble form. Suspensions of the active compounds may be prepared, in some cases, as appropriate oily injection suspensions. Suitable lipophilic solvents or vehicles may include, but are not limited to, fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. The suspension may contain suitable stabilizers. In some embodiments, the suspension comprises agents that increase the solubility of the compounds. The stabilizers or solubility increasers may allow for the preparation of highly concentrated solutions.

The composition may be formulated as a depot preparation in some embodiments. Such long-acting formulations may be formulated, in some cases, with suitable polymeric or hydrophobic materials (for example, as an emulsion in an acceptable oil) or ion exchange resins, oras sparingly soluble derivatives, for example, as a sparingly soluble salt.

The composition may be prepared in the form of a liquid pharmaceutical preparation. In some embodiments, the liquid pharmaceutical preparation comprises a solution, a suspension, or an emulsion. The composition may be included in the liquid pharmaceutical preparation in any of a variety of forms. For example, the composition may be comprised by an aqueous or saline solution (e.g., suitable for inhalation), may be microencapsulated, may be encochleated, may be coated onto microscopic gold particles, may be contained in liposomes, may be nebulized, may be aerosolized, or may be dried onto a sharp object to be scratched into the skin.

The composition may be prepared in the form of a solid pharmaceutical preparation. For example, the composition may be included within granules; powders; tablets; coated tablets; (micro)capsules; suppositories; syrups; emulsions; suspensions; creams; or dropsor preparations with protracted release of active compounds. In some embodiments, the composition is prepared in the form of a solid pharmaceutical preparation configured to be dissolved or otherwise converted to a pharmaceutical liquid preparation. For example, the composition may be prepared in the form of a dry powder that may be dissolved in water to form an aqueous solution. Liquid or solid compositions may include an additive such as an excipient. In some embodiments, the additive comprises disintegrants, binders, coatingagents, swelling agents, lubricants, flavorings, sweeteners or solubilizers, although any of a variety of additives may be used and the disclosure is not so limited.

In some embodiments, a composition may be administered to provide sequential exposures to a composition over a certain time period, for example, hours, days, weeks, months or years.

For example, the composition may be administered by repeated administrations or by controlled release. For example, the composition may be control-released over the time-period such that it sustains a dosage of the composition during that time-period. Control release may be achieved, for example, by oral dosage forms, bolus injections, transdermal patches, subcutaneous implants, or other methods such as those described herein. Maintaining a substantially constant concentration of a composition may be desired in some cases.

Other delivery systems suitable for use in certain embodiments may include time-release, delayed release, sustained release, or controlled release delivery systems. Such systems may advantageously avoid the need for repeated administrations, increasing convenience to the subject. Many types of delivery systems are available and known to those of ordinary skill in the art. The composition may be administered, for example, using a delivery system such as a polymer-based systems such as polylactic and/or polyglycolic acids, polyanhydrides, polycaprolactones and/or combinations of these; nonpolymer systems that are lipid-based including sterols such as cholesterol, cholesterol esters, and fatty acids or neutral fats such as mono-, di- and triglycerides; hydrogel release systems; liposome-based systems; phospholipid based-systems; silastic systems; peptide based systems; wax coatings; compressed tablets using conventional binders and excipients; or partially fused implants. Specific examples include, but are not limited to, erosional systems in which the composition is contained in a form within a matrix, or diffusional systems in which an active component controls the release rate. The formulation may be present as, for example, microspheres, hydrogels, polymeric reservoirs, cholesterol matrices, or polymeric systems, etc. In some embodiments, the system may allow sustained or controlled release of a composition to occur, for example, through control of the diffusion or erosion/degradation rate of the formulation. In addition, a pump-based hardware delivery system may be used in some embodiments.

In some embodiments, the composition is delivered using an implant. The implant may be configured for short-term or long-term release of the composition. In some embodiments, the delivery system is a long-term release implant. Use of a long-term release implant may be particularly suitable in some embodiments. “Long-term release,” as used herein, means that an implant containing a composition as described herein is constructed and arranged to deliver therapeutically effective levels for In some embodiments, the composition is delivered at therapeutically effective levels over a period of greater than or equal to 1 h, greater than or equal to 2 h, greater than or equal to 4 h, greater than or equal to 6 h, greater than or equal to 12 h, greater than or equal to 24 h, greater than or equal to 3 days, greater than or equal to 7 days, greater than or equal to 10 days, greater than or equal to 14 days, greater than or equal to 21 days, greater than or equal to 30 days, greater than or equal to 45 days, greater than or equal to 60 days, greater than or equal to 90 days, or greater. In some embodiments, the composition is delivered at therapeutically effective levels over a period of less than or equal to 365 days, less than or equal to 200 days, less than or equal to 90 days, less than or equal to 60 days, less than or equal to 45 days, less than or equal to 30 days, less than or equal to 21 days, less than or equal to 14 days, less than or equal to 10 days, less than or equal to 7 days, less than or equal to 3 days, less than or equal to 24 h, less than or equal to 12 h, or less. Combinations of these ranges are possible. For example, in some embodiments, In some embodiments, the composition is delivered at therapeutically effective levels over a period of greater than or equal to 1 h and less than or equal to 365 days. Long-term release implants are well known to those of ordinary skill in the art, and include some of the release systems described herein.

In certain embodiments a composition can be combined with a suitable pharmaceutically acceptable carrier. In general, pharmaceutically acceptable carriers suitable for use are well-known to those of ordinary skill in the art. As used herein, a “pharmaceutically acceptable carrier” refers to a non-toxic material that does not significantly interfere with the effectiveness of the biological activity of the active compound(s) to be administered, but is used as a formulation ingredient, for example, to stabilize or protect the active compound(s) within a composition before use. For example the composition may be incorporated into a liposome, incorporated into a polymer release system, suspended in a liquid (e.g., in a dissolved form or a colloidal form), or other methods such as those described herein. The carrier may include one or more compatible solid or liquid fillers, diluents or encapsulating substances which are suitable for administration to a human or other vertebrate animal. The term “carrier” denotes an organic or inorganic ingredient, which may be natural or synthetic, with which one or more active compounds of the disclosure are combined to facilitate application. The carrier may be co-mingled or otherwise mixed with one or more compositions as described herein, and/or with each other, in a manner such that there is no interaction which would substantially impair the desired pharmaceutical efficacy. The carrier may be either soluble or insoluble, depending on the application. Examples of well-known carriers include, but are not limited to, glass, polystyrene, polypropylene, polyethylene, dextran, nylon, amylase, natural and modified cellulose, polyacrylamide, agarose and magnetite. The nature of the carrier may be either soluble or insoluble.

The formulations described herein may be administered in pharmaceutically acceptable solutions, in some embodiments. Pharmaceutically acceptable solutions may routinely contain pharmaceutically acceptable concentrations of salt, buffering agents, preservatives, compatible carriers, adjuvants, emulsifiers, diluents, excipients, chelating agents, fillers, drying agents, antioxidants, antimicrobials, preservatives, binding agents, bulking agents, silicas, solubilizers, stabilizers and optionally other therapeutic ingredients, that may be used with the composition. For example, if the formulation is a liquid, the carrier may be a solvent, partial solvent, or non-solvent, and may be aqueous or organically based. Non-limiting examples of suitable formulation ingredients include diluents such as calcium carbonate, sodium carbonate, lactose, kaolin, calcium phosphate, or sodium phosphate; granulating and disintegrating agents such as cornstarch or algenic acid; binding agents such as starch, gelatin or acacia; lubricating agents such as magnesium stearate, stearic acid, or talc; time-delay materials such as glycerol monostearate or glycerol distearate; suspending agents such as sodium carboxymethylcellulose, methylcellulose, hydroxypropylmethylcellulose, sodium alginate, polyvinylpyrrolidone; dispersing or wetting agents such as lecithin or other naturally-occurring phosphatides; thickening agents such as cetyl alcohol or beeswax; buffering agents such as acetic acid and salts thereof, citric acid and salts thereof, boric acid and salts thereof, or phosphoric acid and salts thereof; or preservatives such as benzalkonium chloride, chlorobutanol, parabens, or thimerosal. The compositions of the disclosure may be formulated into preparations in solid, semi-solid, liquid or gaseous forms such as tablets, capsules, elixirs, powders, granules, ointments, solutions, depositories, inhalants or injectables, etc.

Suitable buffering agents include, but are not limited to: acetic acid and a salt (1-2% w/v); citric acid and a salt (1-3% w/v); boric acid and a salt (0.5-2.5% w/v); and phosphoric acid and a salt (0.8-2% w/v). Suitable preservatives include, but are not limited to, benzalkonium chloride (0.003-0.03% w/v); chlorobutanol (0.3-0.9% w/v); parabens (0.01-0.25% w/v) and thimerosal (0.004-0.02% w/v).

Preparations may include solutions, suspensions, or emulsions as described above. The solutions, suspensions, or emulsions may be sterile. In some embodiments, the solutions, suspensions, or emulsions are aqueous. In some embodiments the solutions, suspensions or emulsions are non-aqueous. The sterile aqueous or nonaqueous solutions, suspensions, or emulsions may be isotonic with the blood of the subject in certain embodiments. Non-limiting examples of nonaqueous solvents are polypropylene glycol, polyethylene glycol, vegetable oil such as olive oil, sesame oil, coconut oil, arachis oil, peanut oil, mineral oil, injectable organic esters such as ethyl oleate, or fixed oils including synthetic mono or di-glycerides. The solution, suspension, or emulsion may include an aqueous carrier. Exemplary aqueous carriers that may be used include, but are not limited to, water, alcoholic/aqueous solutions, emulsions or suspensions, including saline and buffered media. The preparation may further comprise a parenteral vehicle configured to improve suitability of a solution, suspension, or emulsion for parenteral delivery. Parenteral vehicles include sodium chloride solution, 1,3-butandiol, Ringer's dextrose, dextrose and sodium chloride, lactated Ringer's or fixed oils. Intravenous vehicles include fluid and nutrient replenishers, electrolyte replenishers (such as those based on Ringer's dextrose), and the like. Preservatives and other additives may also be present in some embodiments, such as, for example, antimicrobials, antioxidants, chelating agents and inert gases and the like.

In some embodiments, a composition as described herein may be brought into association or contact with a suitable carrier. The suitable carrier may comprise one or more accessory ingredients. The preparation may be prepared by any of a variety of suitable techniques. For example, the preparation may be prepared by uniformly and intimately associating a composition with a liquid carrier, a finely divided solid carrier or both. In some embodiments, the resulting preparation may be shaped (e.g., into a tablet, a pellet, etc.).

The compositions as discussed herein may be administered per se (neat) or in the form of a pharmaceutically acceptable salt. Although non-pharmaceutically acceptable salts may be used for preparing compositions, when used in medicine salts should be pharmaceutically acceptable. The term “pharmaceutically acceptable salts” includes salts of compositions described herein, prepared in combination with, for example, acids or bases. Pharmaceutically acceptable salts can be prepared as alkaline metal salts, such as lithium, sodium, or potassium salts; or as alkaline earth salts, such as magnesium or calcium salts. Examples of suitable bases that may be used to form salts include ammonium, or mineral bases such as sodium hydroxide, lithium hydroxide, potassium hydroxide, calcium hydroxide, magnesium hydroxide, and the like. Examples of suitable acids that may be used to form salts include inorganic or mineral acids such as hydrochloric, hydrobromic, hydroiodic, hydrofluoric, nitric, carbonic, monohydrogencarbonic, phosphoric, monohydrogenphosphoric, dihydrogenphosphoric, sulfuric, monohydrogensulfuric, phosphorous acids and the like. Other suitable acids include organic acids, for example, acetic, propionic, isobutyric, maleic, malonic, benzoic, succinic, suberic, fumaric, mandelic, phthalic, benzenesulfonic, p-tolylsulfonic, citric, tartaric, methanesulfonic, glucuronic, galacturonic, salicylic, formic, naphthalene-2-sulfonic, and the like. Still other suitable acids include amino acids such as arginate, aspartate, glutamate, and the like. Also, such salts can be prepared as alkaline metal or alkaline earth salts, such as sodium, potassium or calcium salts of the carboxylic acid group.

In another aspect, the present disclosure also provides any of the above-mentioned compositions in kits, optionally including instructions for use of the composition for the treatment of a disease or condition (e.g., by targeting a senescent cell, a fat cell, a cancer cell, or a fibroblast). In some cases, the kit can include a description of use of the compositions as discussed herein. The kit also can include instructions for use of a combination of two or more compositions. Instructions also may be provided for administering the compositions by any suitable technique as previously described, for example, forally, intravenously, pump or implantable delivery device, or via another known route of drug delivery.

The kits described herein may also contain one or more containers. The container may contain compositions and other ingredients as previously described. The kits also may contain instructions for mixing, diluting, and/or administrating the compositions of the disclosure in some cases. The kits also can include other containers with one or more solvents, surfactants, preservative and/or diluents (e.g., normal saline (0.9% NaC), or 5% dextrose) as well as containers for mixing, diluting or administering the components in a sample or to a subject in need of such treatment.

The compositions of the kit may be provided as any suitable form, for example, as liquid pharmaceutical preparations or as solid pharmaceutical preparations. When the composition provided is a dry powder, the composition may be reconstituted by the addition of a suitable solvent, which may also be provided in some cases. In embodiments where liquid forms of the composition are used, the liquid form may be concentrated or ready to use. The solvent will depend on the composition and the mode of use or administration. Suitable solvents for drug compositions are well known, for example as previously described, and are available in the literature. The solvent will depend on the composition and the mode of use or administration.

In still another aspect, the disclosure includes the promotion of one or more of the above-described embodiments, e.g., in vitro or in vivo, promotion of treatment or prevention of a tumor, e.g., by administering, to a subject, compositions such as those described herein.

The treatments disclosed herein may be given to any subject, for example, a human, or a non-human animal, such as a dog, a cat, a horse, a rabbit, a cow, a pig, a sheep, a goat, a rat (e.g., Rattus Norvegicus), a mouse (e.g., Mus musculus), a guinea pig, a non-human primate (e.g., a monkey, a chimpanzee, a baboon, an ape, a gorilla, etc.), or the like.

An antibody, as described herein, may comprise a biopolymer, such as a polypeptide. For example, the antibody may comprise a protein. In some embodiments, the antibody is a glycoprotein. However, in some embodiments, the antibody comprising a protein is a glycoprotein. The antibody may be substantially encoded by immunoglobulin genes or fragments of immunoglobulin genes. Any of a variety of immunoglobulin genes or fragments thereof are known to those of ordinary skill in the art. For example, the antibody may be substantially encoded by immunoglobulin genes include the kappa, lambda, alpha, gamma, delta, epsilon, and mu constant region genes, as well as myriad immunoglobulin variable region genes and fragments thereof. The antibody may be encoded by a light chain immunoglobulin gene or a fragment thereof. Light chain immunoglobulins may be classified as either kappa or lambda.

The antibody may be encoded by a heavy chain immunoglobulin gene or a fragment thereof. Heavy chain immunoglobulins may be classified as gamma, mu, alpha, delta, or epsilon (which in turn define the immunoglobulin classes, IgG, IgM, IgA, IgD and IgE, respectively).

An immunoglobulin structural unit may comprise a tetramer. Each tetramer may be composed of two identical pairs of polypeptide chains, each pair having one light chain and one heavy chain.

In some embodiments, the light chain has a molecular weight of greater than or equal to 15 kDa, greater than or equal to 20 kDa, greater than or equal to 25 kDa, or greater. In some embodiments, the light chain has a molecular weight of less than or equal to 35 kDa, less than or equal to 30 kDa, less than or equal to 25 kDa, or less. Combinations of these ranges are possible. For example, in some embodiments, the light chain has a molecular weight of greater than or equal to 15 kDa and less than or equal to 35 kDa.

In some embodiments, the heavy chain has a molecular weight of greater than or equal to 50 kDa, greater than or equal to 55 kDa, greater than or equal to 60 kDa, or greater. In some embodiments, the heavy chain has a molecular weight of less than or equal to 70 kDa, less than or equal to 65 kDa, less than or equal to 60 kDa, or less. Combinations of these ranges are possible. For example, in some embodiments, the heavy chain has a molecular weight of greater than or equal to 50 kDa and less than or equal to 70 kDa.

The N-terminus of each polypeptide chain may define a variable region of the immunoglobulin structural unit. The variable region may be primarily responsible for antigen recognition. In some embodiments, the variable region comprises greater than or equal to 95, greater than or equal to 98, greater than or equal to 100, greater than or equal to 103, greater than or equal to 105, or more amino acids. In some embodiments, the variable region comprises less than or equal to 115, less than or equal to 113, less than or equal to 110, less than or equal to 108, less than or equal to 105, or fewer amino acids. Combinations of these ranges are possible. For example, in some embodiments, the variable region comprises of greater than or equal to 95 and less than or equal to 115 amino acids.

Antibodies may exist as intact immunoglobulins. However, in some embodiments, antibodies exist as any of a number of immunoglobulin fragments.

The immunoglobulin fragment may be produced by digestion with any of a variety of peptidases (e.g., pepsin). For example, in some embodiments immunoglobulin fragments may be formed by digesting an antibody using pepsin. In some, exemplary embodiments, pepsin is used to digest the Fc domain of an antibody, e.g., by degrading disulfide linkages in a hinge region of the Fc domain to produce F(ab)′2. The F(ab)′2 is, according to certain embodiments, a dimer of Fab which itself is a light chain joined to VH-CH1 by a disulfide bond. The F(ab)′2 may be reduced to break the disulfide linkage in the hinge region, thereby converting the (Fab′)₂ dimer into a Fab′ monomer. The Fab′ monomer, according to some embodiments, comprises Fab and a part of the hinge region of the Fc domain.

In some embodiments, the immunoglobulin fragment is synthesized de novo. The immunoglobulin fragment may be produced by any of a variety of methods known to those of ordinary skill in the art, such as by chemical synthesis, by utilizing recombinant DNA methodology, or by “phage display” methods.

Examples of antibodies include single chain antibodies, e.g., single chain Fv (scFv) antibodies in which a variable heavy and a variable light chain are joined together (directly or through a peptide linker) to form a continuous polypeptide. In one embodiment, the antibody is a monoclonal antibody.

In some embodiments, the composition comprising the antibody or antibody fragment is configured such that the antibody or antibody fragment retains a relatively high affinity for a target molecule. Without wishing to be bound by theory, the affinity of the antibody or antibody fragment for the target may be inversely related to the dissociation constant (K_{D}) between the antibody or antibody fragment and the target, so that a lower K_{D} value corresponds to a higher affinity. In some embodiments, the K_{D} value of the antibody or antibody fragment is less than or equal to 10⁻⁶ M, less than or equal to 10⁻⁷ M, less than or equal to 10⁻⁸ M, less than or equal to 10⁻⁹ M, less than or equal to 10⁻¹⁰ M, or less under physiological conditions. In some embodiments, the K_{D} value of the antibody or antibody fragment is greater than or equal to 10⁻¹³ M, greater than or equal to 10⁻¹² M, greater than or equal to 10⁻¹¹ M, or more. Combinations of these ranges are also possible. For example, in some embodiments, the K_{D} value of the antibody or the antibody fragment is greater than or equal to 10⁻¹² M and less than or equal to 10⁻⁶ M. The K_{D} value of the composition may be determined by a test that would be well-known to one of ordinary skill in the art.

In some embodiments, the K_{D} of the composition may be relatively close to the K_{D} of the pure antibody or antibody fragment analogous to the antibody or antibody fragment of the composition. This relatively high affinity of the composition for its target may be associated with the fact that the composition is a fusion protein.

In this context, the term “antibody fragment” can refer to at least one portion of an antibody, that retains the ability to specifically interact with (e.g., by binding, steric hindrance, stabilizing/destabilizing, spatial distribution) an epitope of an antigen. Examples of antibody fragments include, but are not limited to, any antibody fragments described elsewhere herein and including Fab, Fab′, F(ab′)2, Fv fragments, scFv antibody fragments, disulfide-linked Fvs (sdFv), a Fd fragment consisting of the VH and CH1 domains, linear antibodies, single domain antibodies such as sdAb (either VL or VH), camelid VHH domains, multi-specific antibodies formed from antibody fragments such as a bivalent fragment comprising two Fab fragments linked by a disulfide bridge at the hinge region, and an isolated CDR or other epitope binding fragments of an antibody. An antigen binding fragment can also be incorporated into single domain antibodies, maxibodies, minibodies, nanobodies, intrabodies, diabodies, triabodies, tetrabodies, v-NAR and bis-scFv.

In some embodiments, the antibody may be a minibody or a nanobody. In the context of the present disclosure, it has been inventively recognized that use of a minibody or a nanobody may advantageously increase the binding affinity of the antibody, relative to a full-sized antibody.

The following examples are intended to illustrate certain embodiments of the present disclosure, but do not exemplify the full scope of the disclosure.

### Example 1

In this prophetic example, to a human subject 80 years old has muscle weakness, a composition is provided that comprises an antibody that recognizes senescent cells, and an enzyme. In this example, the antibody is one that can be obtained commercially, e.g., a senescent cell-recognizing antibody or a monoclonal antibody.

The enzyme is one that causes leakage of a lysosome, which leads to the production of antigens. In this example, the enzyme is an oxidase that produces reactive oxygen species, which damages or disrupts the lysosome and causes it to leak. The oxidase creates an exemplary reactive oxygen species, a superoxide. In this example, the oxidase is xanthine oxidase.

The xanthine oxidase is connected to the antibody via a covalent bond, and is administered to the subject intravenously or intramuscularly. Upon reaching the composition's target cells, the enzyme reacts with the lysosome, causing damage or disruption, allowing antigens to be created, which boost the immune response of the subject. In such fashion, the immune system of the subject is able to more effectively target the senescent cells of the subject.

### Example 2

In this prophetic example, to a human subject having NASH, a composition is provided that comprises an antibody that recognizes senescent cells, and an enzyme. In some cases, the antibody is one that can be obtained commercially, e.g., a senescent cell-recognizing antibody or a monoclonal antibody.

The enzyme is one that causes leakage of a lysosome, which leads to the production of antigens. In this example, the enzyme is an oxidase that produces reactive oxygen species, which damages or disrupts the lysosome and causes it to leak. The oxidase creates an exemplary reactive oxygen species, a superoxide. In this example, the oxidase is xanthine oxidase.

The xanthine oxidase is connected to the antibody via a covalent bond, and is administered to the subject intravenously or intramuscularly. Upon reaching the composition's target cells the enzyme reacts with the lysosome, causing damage or disruption, allowing antigens to be created, which boost the immune response of the subject. In such fashion, the immune system of the subject is able to more effectively target the senescent cells of the subject.

### Example 3

In this prophetic example, to a human subject having Alzheimer' s disease, a composition is provided that comprises an antibody that recognizes senescent cells, and an enzyme. In some cases, the antibody is one that can be obtained commercially, e.g., a senescent cell-recognizing antibody or a monoclonal antibody.

The enzyme is one that causes leakage of a lysosome, which leads to the production of antigens. In this example, the enzyme is an oxidase that produces reactive oxygen species, which damages or disrupts the lysosome and causes it to leak. The oxidase may be able to create a reactive oxygen species, such as a superoxide. In this example, the oxidase is xanthine oxidase.

The xanthine oxidase is connected to the antibody via a covalent bond, and is administered to the subject intravenously or intramuscularly. Upon reaching the composition's target cells the enzyme reacts with the lysosome, causing damage or disruption, allowing antigens to be created, which boost the immune response of the subject. In such fashion, the immune system of the subject is able to more effectively target the senescent cells of the subject.

### Example 4

In this prophetic example, a human subject is treated as in Example 1, but in addition, the subject is also provided with a treatment agent to withdraw and/or suppress antioxidants, which affects with action of the composition. In one example, the subject is provided with an unsaturated fatty acid, such as CH₂=CHCOOH. The fatty acids acts to inhibit action of the antioxidants, leading to a larger response to the composition.

### Example 5

In this prophetic example, a human subject is treated as in Example 1, but in addition, the subject is exposed to a statin, such as atorvastatin, which may act to inhibit action of the antioxidants, leading to a larger response to the composition.

### Example 6

In this prophetic example, a composition comprising a senescent cell recognizing antibody and a xanthine oxidase enzyme is prepared using an amino-to-amino crosslinking agent. Senescent cells, which are recognized by an antibody, are cultured in 96 well plates and treated with the conjugate at appropriate concentrations from 10 microgram/ml to 100 microgram/mL. Hypoxanthine, an exemplary enzyme substrate of xanthine oxidase, was provided to the cell culture media at 1 mM concentration. Markers of immunogenic cell death (ICD), including Calreticulin exposure, extra-cellular ATP formation, and HMGB1 formation, are assayed at 1 h, 2 h, 4 h, 6 h, 12 h, and 24 h after the provision of hypoxanthine. A graph of the results is shown in FIG. 3. Appropriately calreticulin forms early followed by ATP formation and then followed by HMGB1 formation. This establishes that the cells produce the markers of ICD and are expected in the course of dying to form neoantigens which activate T cells and direct them to the senescent cells.

### Example 7

Once senescent cells are killed in a process which induces ICD the subject bearing the subject is “immunized” to such cells. Thus ICD protects the subject from senescent cells in the future.

To demonstrate this, in this prophetic example, the senescent cells fragments killed in Example 2 are collected and injected into the left flank of BALB/c mice. Eight days after injection, live senescent cells are injected into the right flank of each mouse. Mice are monitored for senescent cells in the right flank for 120 days. Mice treated with ICD dead cells are compared with mice that are injected with saline. At 120 days, 92% of the mice injected with ICD dead cells are senescent cell free while only 7% of saline injected mice are senescent cell free.

### Example 8

In this prophetic example, to a human subject 80 years old has a BMI of 55, a composition is provided that comprises an antibody that recognizes oversized fat cells, and an enzyme. In some cases, the antibody is one that can be obtained commercially, e.g., a fat cell-recognizing antibody or a monoclonal antibody.

The enzyme is one that causes leakage of a lysosome, which leads to the production of antigens. In this example, the enzyme is an oxidase that produces reactive oxygen species, which damages or disrupts the lysosome and causes it to leak. The oxidase creates an exemplary reactive oxygen species, a superoxide. In this example, the oxidase is xanthine oxidase.

The xanthine oxidase is connected to the antibody via a covalent bond, and is administered to the subject intravenously or intramuscularly. Upon reaching the composition's target cells the enzyme reacts with the lysosome, causing damage or disruption, allowing antigens to be created, which boost the immune response of the subject. In such fashion, the immune system of the subject is able to more effectively target the oversized fat cell in the subject. Following treatment after 3 months the subjects BMI is reduced to 37.

### Example 9

In this prophetic example, a conjugate between an oversized fat cell recognizing antibody and the enzyme xanthine oxidase is prepared using an amino-to-amino crosslinking agent. Oversized fat cells, which are recognized by an antibody, are cultured in 96 well plates and treated with the conjugate at appropriate concentrations from 10 microgram/ml to 100 microgram/mL. Hypoxanthine is provided to the cell culture media at 1 mM concentration. Markers of immunogenic cell death (ICD), including Calreticulin exposure, extra-cellular ATP formation, and HMGB1 formation, are assayed at 1 h, 2 h, 4 h, 6 h, 12 h and 24 h. A graph of the results is shown in FIG. 3. Appropriately calreticulin forms early followed by ATP formation and then followed by HMGB1 formation. This establishes that the cells produce the markers of ICD and are expected in the course of dying to form neoantigens which activate T cells and direct them to the oversized fat cells.

### Example 10

In this prophetic example, to a human subject 80 years old has difficulty breathing, a composition is provided that comprises an antibody that recognizes pulmonary fibroblasts, and an enzyme. In some cases, the antibody is one that can be obtained commercially, e.g., a fibroblast-recognizing antibody or a monoclonal antibody.

The enzyme is one that causes leakage of a lysosome, which leads to the production of antigens. In this example, the enzyme is an oxidase that produces reactive oxygen species, which damages or disrupts the lysosome and causes it to leak. The oxidase creates an exemplary reactive oxygen species, a superoxide. In this example, the oxidase is xanthine oxidase.

The xanthine oxidase is connected to the antibody via a covalent bond, and is administered to the subject intravenously or intramuscularly. Upon reaching the composition's target cells the enzyme reacts with the lysosome, causing damage or disruption, allowing antigens to be created, which boost the immune response of the subject. In such fashion, the immune system of the subject is able to more effectively target the fibroblasts of the subject. Following treatment the subject is more able to breath.

### Example 11

Once fibroblasts are killed in a process which induces ICD the animal bearing the fibrotic lungs is “immunized” to that such cells. Thus ICD protects the animal from pulmonary fibrosis in the future.

To demonstrate this, in this prophetic example, the fibroblasts fragments killed in Example 2 are collected and injected into the left flank of BALB/c mice. Eight days after injection, live fibroblasts are injected into the right flank of each mouse. Mice are monitored for pulmonary fibrosis for 120 days. Mice treated with ICD dead cells are compared with mice that are injected with saline. At 120 days, 92% of the mice injected with ICD dead cells are free of pulmonary fibroblasts while only 7% of saline injected mice are free of pulmonary fibroblasts.

### Example 12

In this prophetic example, to a human subject 80 years old has congestive heart failure, a composition is provided that comprises an antibody that recognizes myocardial fibroblasts, and an enzyme. In some cases, the antibody is one that can be obtained commercially, e.g., a fibroblast-recognizing antibody or a monoclonal antibody.

The enzyme is one that causes leakage of a lysosome, which leads to the production of antigens. In this example, the enzyme is an oxidase that produces reactive oxygen species, which damages or disrupts the lysosome and causes it to leak. The oxidase creates an exemplary reactive oxygen species, a superoxide. In this example, the oxidase is xanthine oxidase.

The xanthine oxidase is connected to the antibody via a covalent bond, and is administered to the subject intravenously or intramuscularly. Upon reaching the composition's target cells the enzyme reacts with the lysosome, causing damage or disruption, allowing antigens to be created, which boost the immune response of the subject. In such fashion, the immune system of the subject is able to more effectively target the fibroblasts of the subject. Following treatment the subject's ejection volume is markedly increased.

### Example 13

In this prophetic example, a conjugate between a fibroblast-recognizing antibody and the enzyme xanthine oxidase is prepared using an amino-to-amino crosslinking agent. Fibroblasts, which are recognized by an antibody, are cultured in 96 well plates and treated with the conjugate at appropriate concentrations from 10 microgram/ml to 100 microgram/mL. Hypoxanthine is provided to the cell culture media at 1 mM concentration. Markers of immunogenic cell death (ICD), including Calreticulin exposure, extra-cellular ATP formation, and HMGB1 formation, are assayed at 1 h, 2 h, 4 h, 6 h, 12 h and 24 h. Appropriately calreticulin forms early followed by ATP formation and then followed by HMGB1 formation. This establishes that the cells produce the markers of ICD and are expected in the course of dying to form neoantigens, which activate T cells and direct them to the fibroblast.

### Example 14

This example describes the creation and analysis of two exemplary compositions, comprising a xanthine oxidase enzyme fragment having a C-terminal truncation (“XO”; SEQ ID NO 2) connected to an epidermal growth factor receptor nanobody (“VHH 9G8”; SEQ ID NO 1) suitable for targeting cancer cells. The compositions were connected in the form of a fusion protein. The fusion protein also included a linker region between the XO and the VHH 9G8, the linker region having the amino acid sequence GGGGSGGGGS (SEQ ID NO 3). In one of the exemplary compositions, the xanthine oxidase was located at the N-terminus of the nanobody (“XO-VHH 9G8”; SEQ ID NO 5). In the other exemplary composition, the xanthine oxidase was located at the C-terminus of the nanobody (“VHH 9G8-XO”; SEQ ID NO 4). Appropriate controls isolated VHH 9G8 and isolated XO-were also included in the experiments. Table 1 presents the amino acid sequences associated with each of these species.

TABLE 1
Amino acid sequences associated with various
compositions described herein.

| SEQ ID NO | Sequence |
|---|---|
| 1 | EVQLVESGGGLVQAGGSLRLSCAASGRTFSSYAMGWFRQAPGKEREFVVAINWSSG |
| | STYYADSVKGRFTISRDNAKNTMYLQMNSLKPEDTAVYYCAAGYQINSGNYNFKDY |
| | EYDYWGQGTQVTVSSALEHHHHHH |
| 2 | MTADKLVFFVNGRKVVEKNADPETTLLAYLRRKLGLSGTKLGCGEGGCGACTVML |
| | SKYDRLQNKIVHFSANACLAPICSLHHVAVTTVEGIGSTKTRLHPVQERIAKSHGSQC |
| | GFCTPGIVMSMYTLLRNQPEPTMEEIENAFQGNLCRCTGYRPILQGFRTFARDGGCCG |
| | GDGNNPNCCMNQKKDHSVSLSPSLFKPEEFTPLDPTQEPIFPPELLRLKDTPRKQLRFE |
| | GERVTWIQASTLKELLDLKAQHPDAKLVVGNTEIGIEMKFKNMLFPMIVCPAWIPEL |
| | NSVEHGPDGISFGAACPLSIVEKTLVDAVAKLPAQKTEVFRGVLEQLRWFAGKQVKS |
| | VASVGGNIITASPISDLNPVFMASGAKLTLVSRGTRRTVQMDHTFFPGYRKTLLSPEEI |
| | LLSIEIPYSREGEYFSAFKQASRREDDIAKVTSGMRVLFKPGTTEVQELALCYGGMAN |
| | RTISALKTTQRQLSKLWKEELLQDVCAGLAEELHLPPDAPGGMVDFRCTLTLSFFFKF |
| | YLTVLQKLGQENLEDKCGKLDPTFASATLLFQKDPPADVQLFQEVPKGQSEEDMVG |
| | RPLPHLAADMQASGEAVYCDDIPRYENELSLRLVTSTRAHAKIKSIDTSEAKKVPGFV |
| | CFISADDVPGSNITGICNDETVFAKDKVTCVGHIIGAVVADTPEHTQRAAQGVKITYE |
| | ELPAIITIEDAIKNNSFYGPELKIEKGDLKKGFSEADNVVSGEIYIGGQEHFYLETHCTI |
| | AVPKGEAGEMELFVSTQNTMKTQSFVAKMLGVPANRIVVRVKRMGGGFGGKETRS |
| | TVVSTAVALAAYKTGRPVRCMLDRDEDMLITGGRHPFLARYKVGFMKTGTVVALE |
| | VDHFSNVGNTQDLSQSIMERALFHMDNCYKIPNIRGTGRLCKTNLPSNTAFRGFGGP |
| | QGMLIAECWMSEVAVTCGMPAEEVRRKNLYKEGDLTHENQKLEGFTLPRCWEECL |
| | ASSQYHARKSEVDKFNKENCWKKRGLCIIPTKFGISFTVPFLNQAGALLHVYTDGSVL |
| | LTHGGTEMGQGLHTKMVQVASRALKIPTSKIYISETSTNTVPNTSPTAASVSADLNGQ |
| | AVYAACQTILKRLEPYKKKNPSGSWEDWVTAAYMDTVSLSATGFYRTPNLGYSFET |
| | NSGNPFHYFSYGVACSEVEIDCLTGDHKNLRTDIVMDVGSSLNPAIDIGQVEGAFVQG |
| | LGLFTLEELHYSPEGSLHTRGPSTYKIPAFGSIPIEFRVSLLRDCPNKKAIYASKAVGEP |
| | PLFLAASIFFAIKDAIRAARAQHTGNNVKELFRLDSPATPEKIRNACVDKFTTL |
| 3 | GGGGSGGGGS |
| 4 | EVQLVESGGGLVQAGGSLRLSCAASGRTFSSYAMGWFRQAPGKEREFVVAINWSSG |
| | STYYADSVKGRFTISRDNAKNTMYLQMNSLKPEDTAVYYCAAGYQINSGNYNFKDY |
| | EYDYWGQGTQVTVSSALEHHHHHHGGGGSGGGGSMTADKLVFFVNGRKVVEKNA |
| | DPETTLLAYLRRKLGLSGTKLGCGEGGCGACTVMLSKYDRLQNKIVHFSANACLAPI |
| | CSLHHVAVTTVEGIGSTKTRLHPVQERIAKSHGSQCGFCTPGIVMSMYTLLRNQPEPT |
| | MEEIENAFQGNLCRCTGYRPILQGFRTFARDGGCCGGDGNNPNCCMNQKKDHSVSL |
| | SPSLFKPEEFTPLDPTQEPIFPPELLRLKDTPRKQLRFEGERVTWIQASTLKELLDLKAQ |
| | HPDAKLVVGNTEIGIEMKFKNMLFPMIVCPAWIPELNSVEHGPDGISFGAACPLSIVEK |
| | TLVDAVAKLPAQKTEVFRGVLEQLRWFAGKQVKSVASVGGNIITASPISDLNPVFMA |
| | SGAKLTLVSRGTRRTVQMDHTFFPGYRKTLLSPEEILLSIEIPYSREGEYFSAFKQASR |
| | REDDIAKVTSGMRVLFKPGTTEVQELALCYGGMANRTISALKTTQRQLSKLWKEELL |
| | QDVCAGLAEELHLPPDAPGGMVDFRCTLTLSFFFKFYLTVLQKLGQENLEDKCGKLD |
| | PTFASATLLFQKDPPADVQLFQEVPKGQSEEDMVGRPLPHLAADMQASGEAVYCDDI |
| | PRYENELSLRLVTSTRAHAKIKSIDTSEAKKVPGFVCFISADDVPGSNITGICNDETVFA |
| | KDKVTCVGHIIGAVVADTPEHTQRAAQGVKITYEELPAIITIEDAIKNNSFYGPELKIEK |
| | GDLKKGFSEADNVVSGEIYIGGQEHFYLETHCTIAVPKGEAGEMELFVSTQNTMKTQ |
| | SFVAKMLGVPANRIVVRVKRMGGGFGGKETRSTVVSTAVALAAYKTGRPVRCMLD |
| | RDEDMLITGGRHPFLARYKVGFMKTGTVVALEVDHFSNVGNTQDLSQSIMERALFH |
| | MDNCYKIPNIRGTGRLCKTNLPSNTAFRGFGGPQGMLIAECWMSEVAVTCGMPAEE |
| | VRRKNLYKEGDLTHENQKLEGFTLPRCWEECLASSQYHARKSEVDKFNKENCWKK |
| | RGLCIIPTKFGISFTVPFLNQAGALLHVYTDGSVLLTHGGTEMGQGLHTKMVQVASR |
| | ALKIPTSKIYISETSTNTVPNTSPTAASVSADLNGQAVYAACQTILKRLEPYKKKNPSG |
| | SWEDWVTAAYMDTVSLSATGFYRTPNLGYSFETNSGNPFHYFSYGVACSEVEIDCLT |
| | GDHKNLRTDIVMDVGSSLNPAIDIGQVEGAFVQGLGLFTLEELHYSPEGSLHTRGPST |
| | YKIPAFGSIPIEFRVSLLRDCPNKKAIYASKAVGEPPLFLAASIFFAIKDAIRAARAQHT |
| | GNNVKELFRLDSPATPEKIRNACVDKFTTL |
| 5 | MTADKLVFFVNGRKVVEKNADPETTLLAYLRRKLGLSGTKLGCGEGGCGACTVML |
| | SKYDRLQNKIVHFSANACLAPICSLHHVAVTTVEGIGSTKTRLHPVQERIAKSHGSQC |
| | GFCTPGIVMSMYTLLRNQPEPTMEEIENAFQGNLCRCTGYRPILQGFRTFARDGGCCG |
| | GDGNNPNCCMNQKKDHSVSLSPSLFKPEEFTPLDPTQEPIFPPELLRLKDTPRKQLRFE |
| | GERVTWIQASTLKELLDLKAQHPDAKLVVGNTEIGIEMKFKNMLFPMIVCPAWIPEL |
| | NSVEHGPDGISFGAACPLSIVEKTLVDAVAKLPAQKTEVFRGVLEQLRWFAGKQVKS |
| | VASVGGNIITASPISDLNPVFMASGAKLTLVSRGTRRTVQMDHTFFPGYRKTLLSPEEI |
| | LLSIEIPYSREGEYFSAFKQASRREDDIAKVTSGMRVLFKPGTTEVQELALCYGGMAN |
| | RTISALKTTQRQLSKLWKEELLQDVCAGLAEELHLPPDAPGGMVDFRCTLTLSFFFKF |
| | YLTVLQKLGQENLEDKCGKLDPTFASATLLFQKDPPADVQLFQEVPKGQSEEDMVG |
| | RPLPHLAADMQASGEAVYCDDIPRYENELSLRLVTSTRAHAKIKSIDTSEAKKVPGFV |
| | CFISADDVPGSNITGICNDETVFAKDKVTCVGHIIGAVVADTPEHTQRAAQGVKITYE |
| | ELPAIITIEDAIKNNSFYGPELKIEKGDLKKGFSEADNVVSGEIYIGGQEHFYLETHCTI |
| | AVPKGEAGEMELFVSTQNTMKTQSFVAKMLGVPANRIVVRVKRMGGGFGGKETRS |
| | TVVSTAVALAAYKTGRPVRCMLDRDEDMLITGGRHPFLARYKVGFMKTGTVVALE |
| | VDHFSNVGNTQDLSQSIMERALFHMDNCYKIPNIRGTGRLCKTNLPSNTAFRGFGGP |
| | QGMLIAECWMSEVAVTCGMPAEEVRRKNLYKEGDLTHFNQKLEGFTLPRCWEECL |
| | ASSQYHARKSEVDKFNKENCWKKRGLCIIPTKFGISFTVPFLNQAGALLHVYTDGSVL |
| | LTHGGTEMGQGLHTKMVQVASRALKIPTSKIYISETSTNTVPNTSPTAASVSADLNGQ |
| | AVYAACQTILKRLEPYKKKNPSGSWEDWVTAAYMDTVSLSATGFYRTPNLGYSFET |
| | NSGNPFHYFSYGVACSEVEIDCLTGDHKNLRTDIVMDVGSSLNPAIDIGQVEGAFVQG |
| | LGLFTLEELHYSPEGSLHTRGPSTYKIPAFGSIPIEFRVSLLRDCPNKKAIYASKAVGEP |
| | PLFLAASIFFAIKDAIRAARAQHTGNNVKELFRLDSPATPEKIRNACVDKFTTLGGGGS |
| | GGGGSEVQLVESGGGLVQAGGSLRLSCAASGRTFSSYAMGWFRQAPGKEREFVVAI |
| | NWSSGSTYYADSVKGRFTISRDNAKNTMYLQMNSLKPEDTAVYYCAAGYQINSGNY |
| | NFKDYEYDYWGQGTQVTVSSALEHHHHHH |

Each fusion protein was prepared by cloning into a pCZN4b expression vector and amplification in TOP 10 E. coli. HEK293 cells were cultured and harvested, and their supernatant was collected—each fusion protein was purified from the supernatant by a process of column purification (Ni-NTA column) using a 20 mM imidazole, 50 mM Tris, 300 mM NaCl, pH 8 wash buffer at 1 mL/min. Then the fusion protein was eluted from the column using an elution buffer of 200 mM imidazole, 50 mM tris, 300 mM NaCl, pH 8 at 1 mL/min. The fusion protein was dialyzed with PBS overnight and the protein concentration was determined using a BCA assay. Each fusion protein and control was His-tagged.

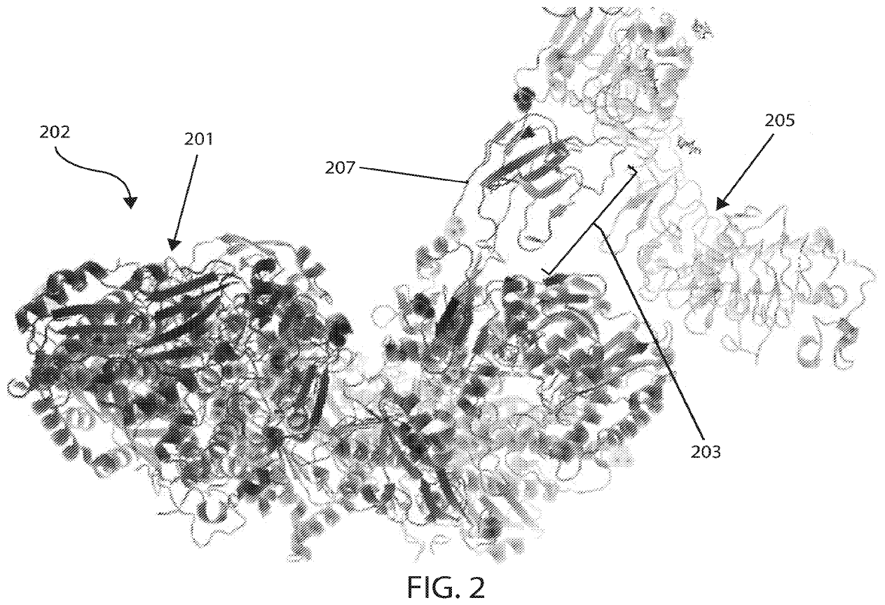

FIG. 2 illustrates the binding of fusion protein 202 to epidermal growth factor receptor 205 (“EGFR”). In FIG. 2, fusion protein 202 is VHH 9G8-XO and comprises nanobody 203 (VHH 9G8) and enzyme fragment 201 (XO), linked by linker 207 (GGGGSGGGGS).

Table 1 reports the dissociation constants (K_{D}), as well as the association rate constant (ka) and dissociation rate constant (ka) associated with binding of each fusion protein and the VHH 9G8 control to EGFR, as determined by surface plasmon resonance (SPR). The surface plasmon resonance was performed using a Biacore 300 Injector with a 120 second contact time, a dissociation time of 240 seconds, and a flow rate of 30 microliters/minute. The results show that the exemplary fusion proteins (XO-VHH 9G8 and VHH 9G8-XO) bind nearly as well as the nanobody control (VHH 9G8). These results demonstrate the usefulness of compositions as described herein, and demonstrate the surprising result that compositions comprising fusion proteins comprising nanobodies, such as XO-VHH 9G8 and VHH 9G8-XO, can have unexpectedly high affinities, comparable to the affinities of the pure antibody.

TABLE 1
Dissociation constants of compositions
comprising EGFR antibodies.

| | K_{D} (M) | kₐ (M⁻¹s⁻¹) | k_{d} (s⁻¹) |
|---|---|---|---|
| VHH 9G8 | 2.85 × 10⁻⁷ | 1.97 × 10⁴ | 5.6 × 10⁻³ |
| VHH 9G8-XO | 1.34 × 10⁻⁷ | 1.77 × 10⁴ | 2.37 × 10⁻³ |
| XO-VHH 9G8 | 4.51 × 10⁻⁸ | 9.54 × 10⁴ | 4.3 × 10⁻³ |

While several embodiments of the present disclosure have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present disclosure. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present disclosure is/are used. Those skilled in the art will recognize, or be able to ascertain using no more than routine experimentation, many equivalents to the specific embodiments of the disclosure described herein. It is, therefore, to be understood that the foregoing embodiments are presented by way of example only and that, within the scope of the appended claims and equivalents thereto, the disclosure may be practiced otherwise than as specifically described and claimed. The present disclosure is directed to each individual feature, system, article, material, and/or method described herein. In addition, any combination of two or more such features, systems, articles, materials, and/or methods, if such features, systems, articles, materials, and/or methods are not mutually inconsistent, is included within the scope of the present disclosure.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

As used herein, “wt %” is an abbreviation of weight percentage. As used herein, “at %” is an abbreviation of atomic percentage.

Some embodiments may be embodied as a method, of which various examples have been described. The acts performed as part of the methods may be ordered in any suitable way. Accordingly, embodiments may be constructed in which acts are performed in an order different than illustrated, which may include different (e.g., more or less) acts than those that are described, and/or that may involve performing some acts simultaneously, even though the acts are shown as being performed sequentially in the embodiments specifically described above.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

In the claims, as well as in the specification above, all transitional phrases such as “comprising,” “including,” “carrying,” “having,” “containing,” “involving,” “holding,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

1. A composition comprising:
a fusion protein comprising an oxidase and at least a portion of a camelid VHH domain antibody,
wherein the fusion protein comprises an amino acid sequence that is at least 70% identical to SEQ ID NO 5.

2. A method of treating a disease or condition, comprising:
administering to a subject a composition comprising a fusion protein comprising an oxidase and at least a portion of a camelid VHH domain antibody,
wherein the fusion protein comprises an amino acid sequence that is at least 70% identical to SEQ ID NO 5.

3-19. (canceled)

20. The composition of claim 1, wherein the camelid VHH domain antibody recognizes a cell, virus, or extracellular material with a K_{D} value below 10⁻⁶ M.

21. The composition of claim 1, wherein the camelid VHH domain antibody recognizes a cell, virus, or extracellular material with a K_{D} value below 10⁻⁷ M.

22. The composition of claim 1, wherein the camelid VHH domain antibody of the composition recognizes a cell, virus, or extracellular material with a K_{D} value smaller than the K_{D} value for a pure camelid VHH domain antibody.

23-48. (canceled)

49. The composition of claim 1, wherein the fusion protein comprises a linker region that has an amino acid sequence that comprises (GGGGS)n, where n is an integer greater than or equal to 1.

50. The composition of claim 1, wherein the amino acid sequence is at least 80% identical to SEQ ID NO 5.

51. The composition of claim 1, wherein the amino acid sequence is at least 90% identical to SEQ ID NO 5.

52. The composition of claim 1, wherein the amino acid sequence is at least 95% identical to SEQ ID NO 5.

53. The method of claim 2, wherein the camelid VHH domain antibody of the composition recognizes a cell, virus, or extracellular material with a K_{D} value below 10⁻⁶ M.

54. The method of claim 2, wherein the camelid VHH domain antibody of the composition recognizes a cell, virus, or extracellular material with a K_{D} value below 10⁻⁷ M.

55. The method of claim 2, wherein the camelid VHH domain antibody of the composition recognizes a cell, virus, or extracellular material with a K_{D} value smaller than the K_{D} value for a pure camelid VHH domain antibody.

56. The method of claim 2, wherein the fusion protein comprises a linker region that has an amino acid sequence that comprises (GGGGS)n, where n is an integer greater than or equal to 1.

57. The method of claim 2, wherein the amino acid sequence is at least 80% identical to SEQ ID NO 5.

58. The method of claim 2, wherein the amino acid sequence is at least 90% identical to SEQ ID NO 5.

59. The method of claim 2, wherein the amino acid sequence is at least 95% identical to SEQ ID NO 5.

60. A composition comprising:
a fusion protein comprising an oxidase and at least a portion of a camelid VHH domain antibody,
wherein the fusion protein comprises an amino acid sequence that is at least 70% identical to SEQ ID NO 4.

61. The composition of claim 60, wherein the fusion protein comprises SEQ ID NO 2.

62. The composition of claim 60, wherein the amino acid sequence is at least 80% identical to SEQ ID NO 4.

63. The composition of claim 60, wherein the amino acid sequence is at least 90% identical to SEQ ID NO 4.
